(19) United States
(12) Patent Application Publication
(10) Pub. No.: US 2024/0115709 A1
(43) Pub. Date: Apr. 11, 2024

(54) ASSEMBLY OF ORGANIC SUPRAMOLECULAR VESSELS FOR CONTROLLED DRUG RELEASE

(71) Applicant: NOBLE PANACEA LABS, INC., Irvine, CA (US)

(72) Inventors: Yassine BELDJOUDI, Irvine, CA (US); Benjie N. LIMKETKAI, Irvine, CA (US); Samer TAHA, Irvine, CA (US)

(73) Assignee: NOBLE PANACEA LABS, INC., Irvine, CA (US)

(21) Appl. No.: 18/277,195

(22) PCT Filed: Feb. 25, 2022
(86) PCT No.: PCT/US2022/017825

§ 371 Date: Aug. 14, 2023

Related U.S. Application Data
(60) Provisional application No. 63/153,533, filed on Feb. 25, 2021.

(51) Int. Cl. A61K 47/40; A61K 8/02; A61K 31/05; A61K 31/355; A61K 31/375

(52) U.S. Cl. CPC A61K 47/40; A61K 8/0245; A61K 31/05; A61K 31/355; A61K 31/375; A61K 2800/56

(57) ABSTRACT

An organic supramolecular structure called organic super molecular vessel (OSMV), including a cyclodextrin host comprising a polymer chain of structural units shaped as a frustoconical annulus formed around a cavity defined from a first end to a second end; and a fatty acid ester bound to at least one of the structural units.

### CROSS-REFERENCE TO RELATED APPLICATION

This application claims priority, and claims the benefit of, U.S. Provisional Patent Application No. 63/153,533 filed on Feb. 25, 2021, the content of which is hereby incorporated herein by its entirety.

### BACKGROUND OF THE INVENTION

### Field of the Invention

Embodiments of the present invention relate to synthesis of organic super molecular vessels (OSMV), OSMV-active ingredient complexes, and formulation including active ingredient and OSMV-active ingredient formulations. In some embodiments of the present invention, delivery of active ingredients is prolonged by delivery of active ingredient from OSMV-active ingredient complexes. By way of example, some embodiments of the present invention relate to external delivery of macromolecules to skin cells through topical application of OSMV-active ingredient formulations and related cosmetic products. Further embodiments of the present invention include pharmaceutical products comprising OSMV-active ingredient formulations for delivery of extended-release pharmaceuticals internally and corresponding methods of treatment with such pharmaceutical formulations.

### SUMMARY

Embodiments of the present invention relate to organic supramolecular structures called organic super molecular vessels (OSMVs), including a cyclodextrin host comprising a polymer chain of structural units shaped as a frustoconical annulus formed around a cavity defined from a first end to a second end; and a fatty acid ester bound to at least one of the structural units.

Additional embodiments of the present invention relate to a method of administering an active ingredient/drug, including preparing an OSMV, loading the OSMV with the active ingredient, applying the loaded OSMV to a patient, and raising the application to biological temperature.

Further embodiments of the present invention relate to a method of administering an active ingredient/drug, including preparing an OSMV, loading the OSMV with the active ingredient, applying the loaded OSMV to a patient, and raising the application to biological temperature.

### DESCRIPTION OF THE DRAWINGS

FIGS. 1a-1d depict illustrations of example cyclodextrins (CDs) and their corresponding tertiary structures in accordance with the principles of the present invention.

FIG. 1e depicts an optimized molecular structure cyclodextrin modified with fatty acid chains in accordance with the principles of the present invention.

FIG. 2a depicts an illustration of the synthesis of CD oleoyl esters (CDOs), α-CDO and β-CDO, which possess 24 fatty acid chains, in accordance with the principles of the present invention.

FIG. 2b depicts an illustration of the synthesis of γ-CDO which possesses 16 fatty acid chains in accordance with the principles of the present invention.

FIG. 3 depicts the FT-IR spectra of γ-CD, γ-CDP (CD palmitoyl ester), and γ-CDO in accordance with the principles of the present invention.

FIG. 4 depicts a thermogravimetric analysis of the γ-CD, γ-CDO and γ-CDP in accordance with the principles of the present invention.

FIG. 5a depicts a ¹H NMR Spectra recorded in H₂O show changes in chemical shifts of γ-CD upon increasing the concentration of niacinamide (NA) in accordance with the principles of the present invention.

FIG. 5b depicts a ¹H NMR Spectra of the γ-CDO and NA recorded in deuterated chloroform (CDCl₃) show changes in chemical shifts upon increasing the concentration of NA in accordance with the principles of the present invention.

FIG. 6a depicts a graph of the dynamic viscosity of the γ-CDO and NA⊂γ-CDO as function of temperature in accordance with the principles of the present invention.

FIG. 6b depicts a graph of the dynamic viscosity of α-tocopherol⊂γ-CDO as function of temperature in accordance with the principles of the present invention.

FIG. 6c depicts a graph of the dynamic viscosity of the β-CDO-1 and β-CDO-2 as function of temperature in accordance with the principles of the present invention.

FIG. 7 depicts a pictogram representation of columnar discotic superstructures in accordance with the principles of the present invention.

FIG. 8 depicts the powder X-ray diffraction patterns of the NA, NA⊂γ-CDO, NA⊂β-CDO and NA⊂γ-CDO in accordance with the principles of the present invention.

FIG. 9 depicts the powder X-ray diffraction patterns of the α-CDO, β-CDP and γ-CDP in accordance with the principles of the present invention.

FIG. 10 depicts the powder X-ray diffraction patterns of the NA, NA⊂γ-CDO, NA⊂β-CDP and NA⊂γ-CDP in accordance with the principles of the present invention.

FIG. 11(a) depicts the kinetics of resveratrol release from the Resveratrol⊂γ-CDO complex in accordance with the principles of the present invention.

FIG. 11(b) depicts the UV-visible absorption spectra collected at room temperature plot of the intensity of absorbance at 315 nm as a function of time in accordance with the principles of the present invention.

FIG. 11(c) depicts a graph of the kinetic of the resveratrol release from the Resveratrol⊂γ-CDO composite in ethanol and water monitored using UV-visible spectroscopy at 315 nm in accordance with the principles of the present invention.

FIG. 11(d) depicts a graph of the kintic of the release of α-tocopherol from α-tocopherol⊂γ-CDO composite monitored using UV-visible spectroscopy at 295 nm in accordance with the principles of the present invention.

FIG. 11(e) depicts a graph of the release kinetic of the magnesium ascorbyl phosphate (MgAsc) from the MgAsc⊂γ-CDO composite in deionized water. The concentration of MgAsc was monitored by UV-vis absorption spectroscopy at 260 nm in accordance with the principles of the present invention.

FIG. 11(f) depicts a graph of diffusion of the Niacinamide from the γ-CDO using the Franz diffusion cell where the concentration of Niacinamide is monitored by UV-Visible spectroscopy experiment was conducted at 23° C. in accordance with the principles of the present invention.

FIG. 11(g) depicts a graph of diffusion of the Niacinamide from the γ-CDO using the Franz diffusion cell where the concentration of Niacinamide is monitored by UV-Visible spectroscopy experiment was conducted at 37° C. in accordance with the principles of the present invention.

FIG. 11(h) depicts a graph of the diffusion kinetics of niacinamide (NA) from the NA⊂γ-CDO complex using Franz diffusion cell at 23 (lower trend) and 37° C. (upper trend) in H₂O in accordance with the principles of the present invention. The concentration of the NA was monitored by absorption spectroscopy at the wavelength of 260 nm.

FIG. 11(i) depicts a graph of the diffusion kinetics of niacinamide (NA) alone using Franz diffusion cell at 23 (lower trend) and 37° C. (upper trend) in H₂O in accordance with the principles of the present invention. The concentration of the NA was monitored by absorption spectroscopy at the wavelength of 260 nm.

FIG. 11(j) depicts a graph of the absorption spectra of the Ginseng, Gotu Kola and Green Tea water extract at concentration of 0.4 mg/L in accordance with the princinples of the present invention.

FIG. 11(k) depicts a graph of the kinetic of the drug release in deionized water in accordance with the princinples of the present invention.

FIG. 11(l) depicts a graph of the diffusion kinetics of resveratrol using Franz diffusion cell at 37° C. in EtOH. The concentration of the resveratrol was monitored by absorption spectroscopy at the wavelength of 315 nm. Diffusion of resveratrol from the resveratrol⊂γ-CDO complex (lower line), and diffusion of the resveratrol (upper line) in accordance with the principles of the present invention.

FIG. 11(m) depicts a graph of UV-Visible absorption spectrum of the Orange Acridine hydrochloride (OA) in H₂O in accordance with the principles of the present invention.

FIG. 11(n) depicts a graph of the diffusion kinetics of the OA through a membrane in the Franz diffusion cell for both pure OA and the OA⊂γ-CDO composite in accordance with the principles of the present invention.

FIG. 11(o) depicts pictures illustrating the change of the color of the solution in the Franz diffusion cell after 4 hours for the diffusion of pure OA while the diffusion of OA from OA⊂γ-CDO is significantly slower in accordance with the principles of the present invention.

FIG. 11(p) depicts the temperature dependance of the OA release from the OA⊂γ-CDO composite in water in accordance with the principles of the present invention.

FIG. 12 depicts the synthesis reaction of the oligosaccharide oleoyl esters in accordance with the principles of the present invention.

FIG. 13 depicts the ¹H NMR spectrum of the α-CDO in CDCl₃ at 298K in accordance with the principles of the present invention.

FIG. 14 depicts the ¹H NMR spectrum of the β-CDO in CDCl₃ at 298K in accordance with the principles of the present invention.

FIG. 15 depicts the synthesis of the γ-CD oleoyl esters in accordance with the principles of the present invention.

FIG. 16 depicts the ¹H NMR spectrum of the γ-CDO in CDCl₃ at 298K in accordance with the principles of the present invention.

FIG. 17 depicts 2D NMR DOSY spectrum of the γ-CDO in CDCl₃ at 298K in accordance with the principles of the present invention.

FIG. 18 depicts the ¹³C NMR spectrum of the γ-CDO in CDCl₃ at 298K in accordance with the principles of the present invention.

FIG. 19 depicts the Synthesis of the cyclodextrin palmitoyl esters in accordance with the principles of the present invention.

FIG. 20 depicts the ¹H NMR spectrum of the α-CDP in CDCl₃ at 298K in accordance with the principles of the present invention.

FIG. 21 depicts the ¹H NMR spectrum of the Oligosaccharide fatty acid ester in CDCl₃ at 298K in accordance with the principles of the present invention.

FIG. 22 depicts the ¹H NMR spectrum of the γ-CDP ester in CDCl₃ at 298K. (PCD) in accordance with the principles of the present invention.

FIG. 23 depicts the 2D NMR DOSY spectrum of the γ-CDP in CDCl₃ at 298K PCD in accordance with the principles of the present invention.

FIG. 24 depicts the thermogravimetric analysis of the α-CDP in accordance with the principles of the present invention.

FIG. 25 depicts the FT-IR spectra of α-CDP, β-CDP and γ-CDP in accordance with the principles of the present invention.

FIG. 26 depicts the FT-IR spectra of α-CDO, β-CDO and γ-CDO in accordance with the principles of the present invention.

### DETAILED DESCRIPTION OF THE PREFERRED EMBODIMENTS

The following description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein, but are to be accorded the full scope consistent with each claim's language, wherein reference to an element in the singular is not intended to mean “one and only one” unless specifically so stated, but rather “one or more.” Similarly, references to an element in the singular in the description mean “one or more” unless specifically stated otherwise. All structural and functional equivalents to the elements of the various embodiments described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.”

With respect to the present application, “about” or “approximately” means within plus or minus one at the last reported digit. For example, about 1.00 means 1.00±0.01 unit.

With respect to the present application, “around” used in conjunction with a numeral measurement means within plus or minus one unit. For example, around 50% means 49%-51%. For example, around 11.01 units means 10.01-12.01.

With respect to the present description “and” and “or” shall be construed as conjunctively or disjunctively, whichever provides the broadest disclosure in each instance of the use of “and” or “or.”

With respect to the present description, “active ingredient” means a pharmaceutical active ingredient, a drug, an antioxidant, a nutrient, a cosmetic active ingredient, and/or a fragrance active ingredient unless otherwise further specified.

With respect to the present description, “active pharmaceutical ingredient” means lidocaine, naproxen, lansoprazole, ibuprofen, acetaminophen, diclofenac, oxycodone, fentanyl, hydrocodone, opioids, chemotherapeutic drugs, letrozole, sonidegib, ruxolitinib, abiraterone, altretamine, Palbociclib, procarbazine, and sunitinib.

With respect to the present description, “active cosmetic ingredient” means alpha or beta hydroxy acid, anti-wrinkle agent, anti-aging agent, skin-lightening agent, anti-dark circle agent, peptide, amino acid, plant extracts, vitamin, antioxidant, anti-inflammatory agent, humectant, keratolytic agent, antibacterial agent, antifungal agent, a sunscreen agent, of which may include but not limited to niacinamide and resveratrol, glycolic acid, lactic acid, salicylic acid, gluconolactone, lactobionic acid, citric acid, hyaluronic acid, sodium hyaluronate, retinol, retinyl palmitate, panthenol, allantoin, ceramide, caffeine, ubiquinone, kojic acid, hydroquinone, ascorbic acid, ascorbyl glucoside, sodium ascorbyl phosphate, magnesium ascorbyl phosphate, acetyl hexapeptide-8, acetyl hexapeptide-3, palmitoyl tripeptide-38, palmitoyl tripeptide-1, palmitoyl tripeptide-5, hydrolyzed rice protein, bakuchiol, Camellia sinensis leaf extract, Centella asiatica extract, Citrus aurantium dulcis fruit extract, Citrus limon fruit extract, ferulic acid, Ginkgo biloba leaf extract, glyceryl linoleate, glyceryl linolenate, Lyceum barbarum fruit extract, oat amino acids, tocopherol, tocopheryl acetate, Vitis vinifera leaf extract, lipoic acid, folic acid, Coffea arabica seed extract, and Cucumis sativus fruit extract.

With respect to the present description, “active fragrance ingredient” means essential oils, fragrance oils, and/or specific aroma compounds such as esters, linear terpenes, cyclic terpenes, and aromatic. Example of esters include geranyl acetate (rose), methyl butyrate (apple), ethyl butyrate (orange), and benzyl acetate (strawberries). Examples of linear terpenes include nerol (neroli), citral (lemongrass), linalool (lavendar), and ocimene (mango). Examples of cyclic terpenes include limonene (orange), camphor (campor laurel), menthol, jasmone (jasmine), and eucalyptol (eucalyptus). Examples of aromatic include eugenol (clove), benzaldehyde (almond), vanillin (vanilla), and thymol (thyme).

Embodiments of the invention relate to synthesis of Organic Super Molecular Vessels (OSMV), preparation of formulations comprising OSMV-active ingredient complexes, and delivery of active ingredients thereby. More particularly, embodiments of the invention relate to external delivery of macromolecules to skin cells through topical application and related cosmetic products through OSMVs. Further embodiments of the present invention include pharmaceutical products comprising OSMVs for delivery of pharmaceuticals internally and methods of treatment with such pharmaceutical compositions. In internally released embodiments, enzymes in saliva and/or in the stomach may break down the OSMV to release the active pharmaceutical ingredient over time. OSMV-active ingredient complexes may comprise a pharmaceutical active ingredient, a cosmetic active ingredient, and/or a fragrance active ingredient.

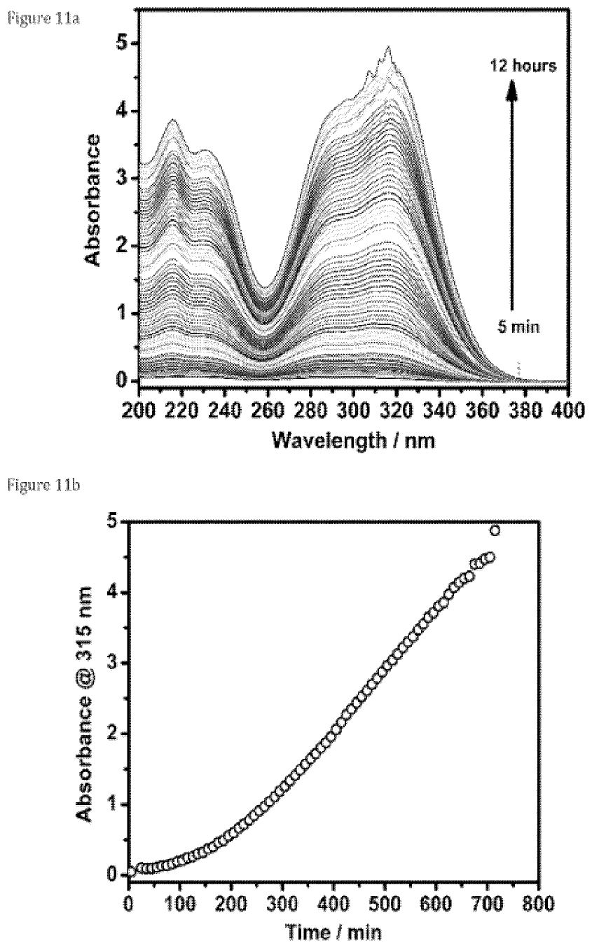

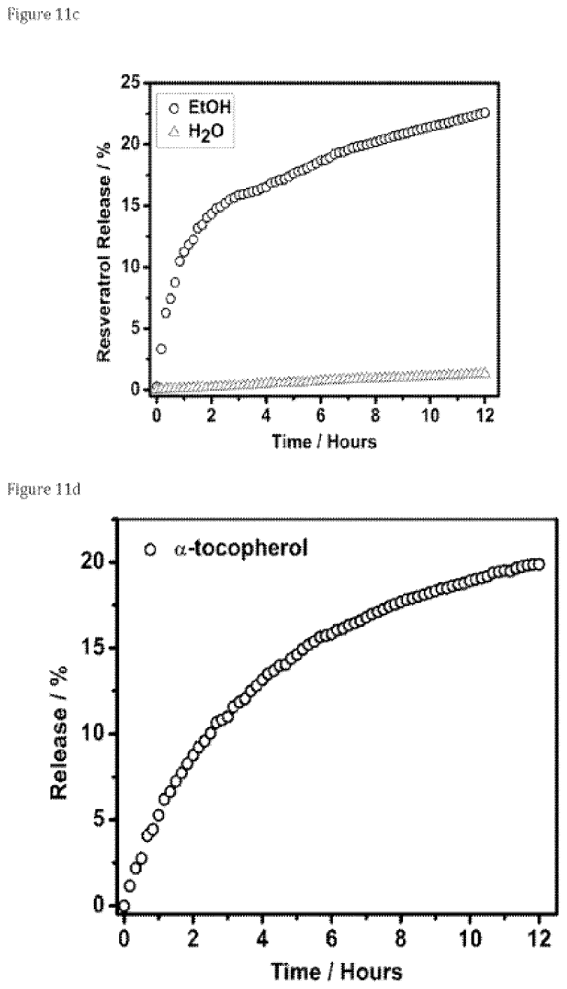

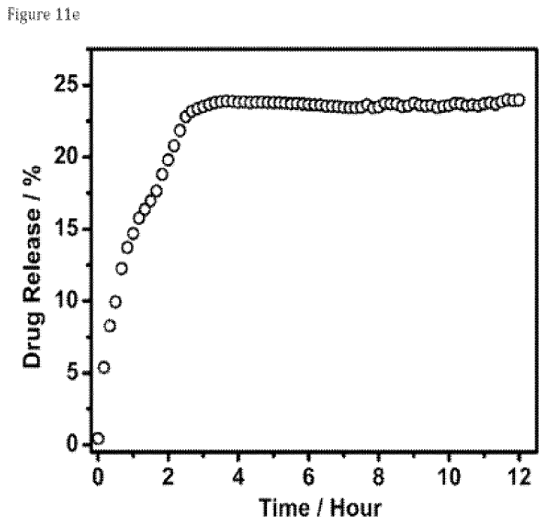

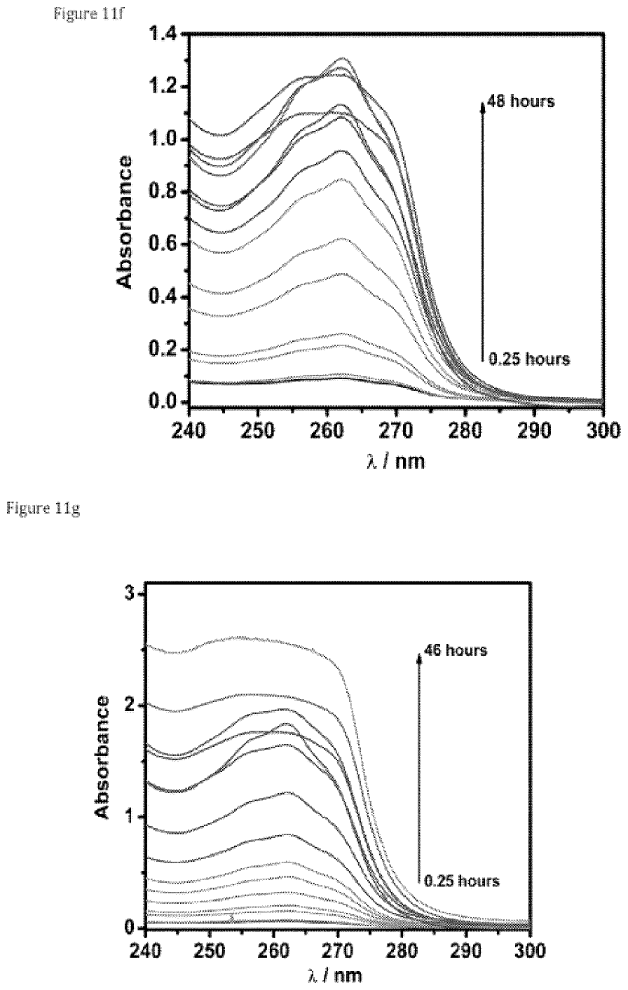

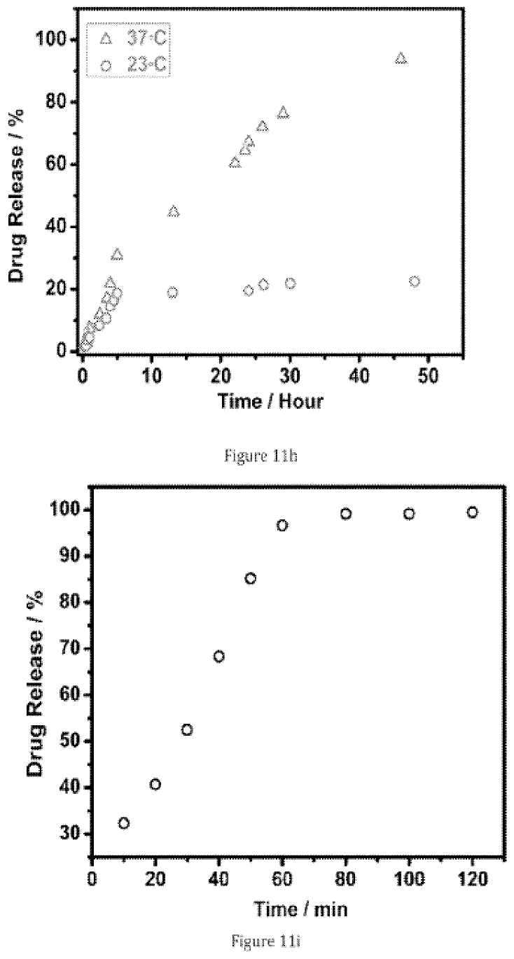

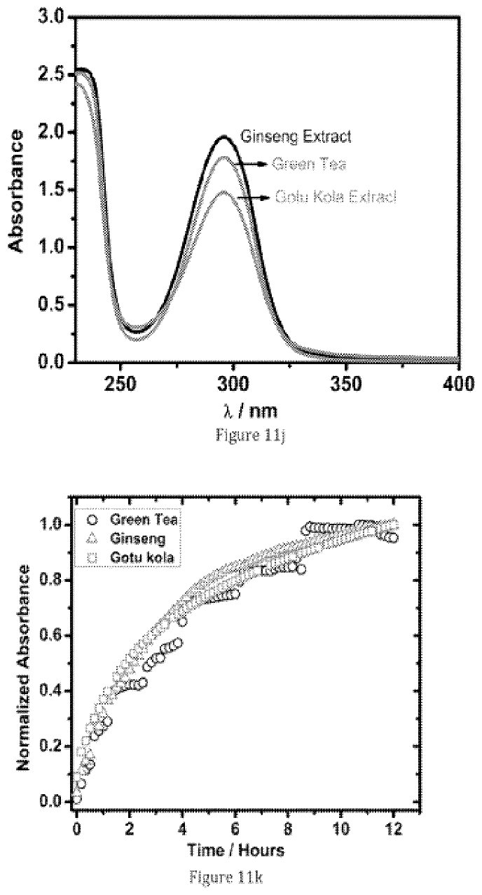

FIG. 11b shows about 12 hours of cumulative release that simulates water dissolution of the loaded OSMV particle. Some examples for active pharmaceutical ingredients (API) that may be loaded into OSMVs include but are not limited to lidocaine, naproxen, lansoprazole, ibuprofen, acetaminophen, diclofenac, oxycodone, fentanyl, and hydrocodone. In some embodiments, controlling the release of API, e.g., opioid pain relief drugs, the release of the dosage can be optimally controlled to provide therapeutic effect at lower dosage to minimize chances of addiction. Furthermore, in embodiment comprising topical application, a high dosage spread over long time with minimum release can achieve long skin therapeutic effect and minimize risk of skin irritation due to overexposure or sensitization of the active ingredient. Oral chemotherapy can also be done using OSMV encapsulated chemotherapeutic drugs. Some examples include letrozole, sonidegib, ruxolitinib, abiraterone, altretamine, Palbociclib, procarbazine, and sunitinib. Dosing may be critical for oral chemotherapy and medication can be less effective if the patient misses a pill or takes pills too close together. Incorrect dosing may have severe side effects. OSMV release of APIs can be beneficial because a dosage can be controlled for steady release for long duration after taking only 1 pill.

Embodiments of the invention include synthesis of OSMVs, further embodiments of the invention include use of OSMVs, including delivery of active ingredients using OSMVs. Delivery of active ingredients with timely precision, such as delayed, or extended release, through OSMVs to the skin cells may maximize skincare product efficacy. FIG. 11b illustrates an example time profile of an OSMV-resveratrol complex, wherein release may not necessarily occur until about 100 minutes, followed by steadily increasing release for about 600 minutes. The release may stop (e.g., 100% cumulative release) at around the 700-minute mark. This is only one OSMV-resveratrol encapsulation. Further different types of encapsulation of OSMV-resveratrol may modify this profile. For example, between a smaller OSMV particle and a bigger OSMV particle, the smaller may release faster than the bigger one.

For example, such OSMVs may comprise cyclic carbohydrates modified with saturated and non-saturated fatty acids. Example fatty acids modifiers include any fatter ester. Examples of natural and readily available fatter esters include at least palmitoyl, oleoyl, oleic, linoleic, linolenic, and stearic acids. Example topical cosmetic active ingredients can be an alpha or beta hydroxy acid, anti-wrinkle agent, anti-aging agent, skin-lightening agent, anti-dark circle agent, peptide, amino acid, plant extracts, vitamin, antioxidant, anti-inflammatory agent, humectant, keratolytic agent, antibacterial agent, antifungal agent, a sunscreen agent, of which may include but not limited to niacinamide and resveratrol, glycolic acid, lactic acid, salicylic acid, gluconolactone, lactobionic acid, citric acid, hyaluronic acid, sodium hyaluronate, retinol, retinyl palmitate, panthenol, allantoin, ceramide, caffeine, ubiquinone, kojic acid, hydroquinone, ascorbic acid, ascorbyl glucoside, sodium ascorbyl phosphate, magnesium ascorbyl phosphate, acetyl hexapeptide-8, acetyl hexapeptide-3, palmitoyl tripeptide-38, palmitoyl tripeptide-1, palmitoyl tripeptide-5, hydrolyzed rice protein, bakuchiol, Camellia sinensis leaf extract, Centella asiatica extract, Citrus aurantium dulcis fruit extract, Citrus limon fruit extract, ferulic acid, Ginkgo biloba leaf extract, glyceryl linoleate, glyceryl linolenate, Lyceum barbarum fruit extract, oat amino acids, tocopherol, tocopheryl acetate, Vitis vinifera leaf extract, lipoic acid, folic acid, Coffea arabica seed extract, and Cucumis sativus fruit extract. OSMV can also encapsulate fragrance ingredients for perfume and fragrance cosmetic applications. Instead of releasing ingredients into the skin below, the encapsulated ingredients inside OSMV are released into the air above. OSMV can encapsulate essential oils, fragrance oils, and/or specific aroma compounds such as esters, linear terpenes, cyclic terpenes, and aromatic. Example of esters include geranyl acetate (rose), methyl butyrate (apple), ethyl butyrate (orange), and benzyl acetate (strawberries). Examples of linear terpenes include nerol (neroli), citral (lemongrass), linalool (lavendar), and ocimene (mango). Examples of cyclic terpenes include limonene (orange), camphor (campor laurel), menthol, jasmone (jasmine), and eucalyptol (eucalyptus). Examples of aromatic include eugenol (clove), benzaldehyde (almond), vanillin (vanilla), and thymol (thyme).

The synthesis of OSMV was achieved in high yield following a procedure using the palmitoyl and oleoyl chloride derivatives which react with the hydroxyl groups of α, β, and γ-cyclodextrin macrocycles (CDs). Regarding API molecular size, APIs may fit within the OSMV scaffold. Regarding molecules that get trapped inside the CD cavity, typically, small molecules that can fit inside the cavity of the CD may fit within the OSMVs. However, CDs can also trap a portion of the molecule without needing to fit the whole molecule inside the CD cavity. OSMV does not have fundamental limitation for hydrophobicity/hydrophilicity. They can encapsulate both water and/or water-insoluble ingredients. However, from a loading efficiency point of view, for active ingredients that are only water soluble and cannot dissolve in other solvents, loading in 100% water may become inefficient if OSMV is allowed to agglomerate in aqueous solution. Such loading is possible, although not necessarily at maximum loading efficiency.

Crystallographic studies revealed that the CD oleoyl esters (CDOs) may be semicrystalline organogels and may behave like discotic liquid crystals, whilst the CD palmitoyl esters (CDPs) may comprise crystalline powders at room temperature but may melt at ˜35° C. Both CDOs and CDPs may form stacked superstructures with interlamellar distances in a range of 3.7-4 nm, suggesting the formation of supramolecular nanotubes. The OSMVs may be soluble in organic solvents which may allow loading medicinal active ingredients in organic solvents while the drug release may be achieved in aqueous media. The non-water-soluble nature of the OSMVs may play a role of shield to slow down the solubility of the active ingredients in water and the cavity of the OSMV CDs may serve as a channel for hosting and releasing guest molecules. Study of the rheology of the CDOs and CDO loaded with niacinamide (NA), NA⊂CDO, revealed that both possess strong gelation behavior at ambient condition while the viscosity may decrease significantly at the biological temperatures (˜37° C.), a behavior which triggers the drug release. Thus, controlling the temperature of synthesis and loading of OSMVs, along with oligosaccharide fatty acid esters which may possess intrinsic cavity to host medicinal active ingredients, may offer numerous opportunities in the drug delivery technologies.

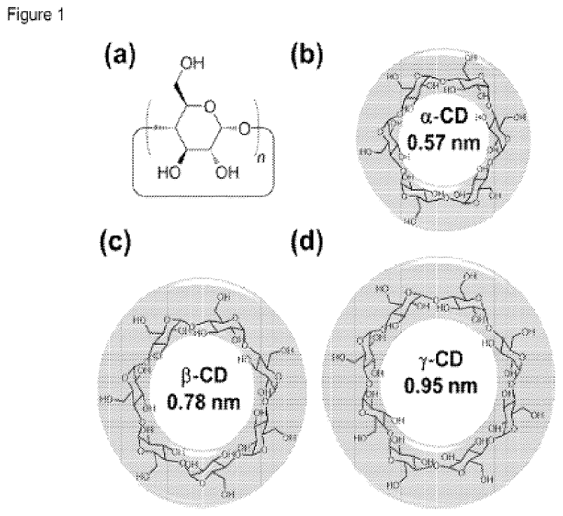

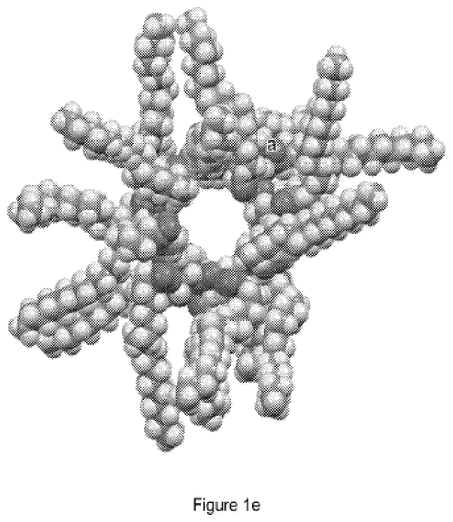

FIG. 1a depicts an illustration of example cyclodextrins (CDs) and their corresponding tertiary structures in accordance with the principles of the present invention.

Carbohydrates (oligosaccharides and/or polysaccharides) have been well-recognized as edible materials. Together with lipids, proteins, and nucleic acids, they are one of the four major classes of biomolecules. Carbohydrates are unique candidates for drug-carrier preparation since they possess several advantages, such as biocompatibility and biodegradability. Cyclodextrins (CD) comprising cyclic oligosaccharides, such as those manufactured by glucosyltransferase degradation of starch, may be useful for biological delivery purposes. OSMVs may be formed by modifying oligosaccharides with fatty acid esters. Examples of suitable oligosaccharides may include beta-cyclodextrin (β-CD), gamma-cyclodextrin (γ-CD), alpha-cyclodextrin (α-CD), maltotriose, stachyose, raffinose, and alpha glucan oligosaccharides. Examples of fatty acids may include at least palmitoyl, oleoyl, and linoleoyl. CDs (cyclodextrins) are used for OSMV preparation by way of example, but embodiments of the present invention are not necessarily limited to modification of cyclodextrins.

The classical CD series are constituted of six (α-CD), seven (β-CD), or eight (γ-CD) asymmetric α-1,4-linked d-glucopyranosyl residues, forming a typical “cage” molecule of a truncated cone shape (FIG. 1) with a hydrophobic cavity and a hydrophilic surface. The diameters of α-, β- and γ-CDs are reported to be 0.57, 0.78, and 0.95 nm, respectively which allows them to form non-covalent host-guest (H-G) inclusion complexes. Thus, these sizes may be the sizes of the internal cavity of the CDs. OSMVs are not limited to preparation with CDs. However, in embodiments wherein OSMVs are made from CDs, the CDs have an added benefit of being able to trap molecules (or parts of molecules) within this internal cavity. The majority of the API molecules may be trapped within the internal cavity of the OSMV in some embodiments. OSMV may be likened to a mesh of interconnected CDs and fatty acid chains that scaffold similar to an interconnected mesh. In some embodiments, the majority of API molecules may be trapped within this scaffold structure.

In addition, in the presence of alkali metals (Li⁺, Na⁺, K⁺), the CDs are linked by coordination of the secondary face hydroxyl groups on alternating d-glucopyranosyl residues to one of the alkali metal cations to form porous cyclodextrin-based Metal organic frameworks (CD-MOFs). For instance, the γ-CD in presence of K⁺, may form a body-centered cubic extended structures with γ-CDs constitute six sides of the cube (CD₆) in the asymmetric unit. Porous CD-MOF may comprise two main types of cages in the molecular structure: (i) spherical voids with diameter of 1.7 nm formed by six γ-CDs, and (ii) cavities with diameter of 0.8 nm formed by face-to-face γ-CD pairs. The size of the CDs may not necessarily determine the size of the molecule loaded. Rather, the size of molecules loaded may be determined by the fatty acid length and density and the scaffold forming the internal web with a porous internal cavity.

The large cavity the CD-MOFs can host drugs and pharmaceutics for controlled release at specific conditions for the benefit of improving product efficacy and safety. However, the rapid dissolution of the CDMOF in aqueous media may render their use in pharmaceutical and cosmetic formulations very challenging to achieve. On the other hand, OSMVs of the present invention may provide the benefits of CD-MOFs but may not necessarily readily dissolve in water.

At the molecular level, the ability of CDs to incorporate small hydrophobic molecules in their cavities confers to the complex formed some different physicochemical properties compared to those of the guest molecule alone. For instance, CDs may stabilize labile guests against oxidation by tailoring the physical properties of guest molecules (e.g., volatility and sublimation properties). The use of CD in drug formulation was proposed since early 1950s, and subsequently, the utilization of CDs in pharmaceutical applications was continually increased as a result of the increase of the bioavailability of several poorly water soluble pharmaceutically active compounds.

New vectors based on cyclodextrins decorated with aliphatic chains may be used in pharmaceutical formulations. Such macrocyclic amphiphiles may form micelles, vesicles, and nanoparticles. In particular, carbohydrate fatty acid esters may be useful in food, cosmetic, and pharmaceutical industries as non-toxic and biodegradable surfactants. CD acylation may be achieved using proteinase N, subtilisin, or lipase. For example, an efficient one-step-catalyzed transesterification of various cyclodextrins by vinyl-acyl fatty esters may be achieved in the presence of thermolysin. For instance, amphiphilic CDs obtained by grafting aliphatic chains on the primary or secondary face exhibit organization properties yielding stable nanospheres or nanocapsules. Although carbohydrate fatty acids may present a large margin of safety in dermal applications and may provide addition stability to oxygen sensitive drugs, carbohydrate fatty acid usage as vesicles for drug delivery in topical cosmetics products remains rare.

The preparation of the amphiphilic cyclodextrins has been a subject of extensive studies over the last 10 years. Most of the efforts has been focused on the preparation of mono- or poly-substituted cyclodextrins on only one face of the cyclodextrin conical shape. The substitution of all the hydroxyl groups on both faces of the cyclodextrins has been rarely explored and often is limited to hydrophilic chains or short alkyl chains such as methyl, butyl and hexyl aliphatic groups. All these compounds form solid powders and to the best of our knowledge no gels has been reported using amphophilic cyclodextrins. In addition, the amphiphilic cyclodextrins have been obtained through the chemical modification of the hydroxyl groups towards thiol (—SH) or primary amine (—NH2) functional groups limiting therefore a large-scale production. The esterification of the cyclodextrins is often achieved through enzymatic catalysis using theromolysin enzyme. The CDs esterification using acyl chloride in one pot-synthesis such oleoyl chloride derivatives has not be reported to the best of our knowledge.

All the examples so far reported of the amphiphilic CDs substituted on both faces of the conical CDs shape uses both hydrophobic and hydrophilic anchors in order to maintain the solubility in aqueous media. For instance, the introduction of hydrophilic oligo(ethylene glycol) chains onto the secondary face of the cyclodextrin ring has enhanced the aqueous solubility of the novel amphiphilic derivatives.

It is worth noting that most of the amphiphilic CDs are based on the β-CD and little research has been carried on the γ-CDs.

Embodiments of the present invention include substitution of both and/or all faces of the CDs using aliphatic chains of >C16 using both saturated and unsaturated biocompatible fatty acids. While CDs decorated with saturated aliphatic chains offers crystalline solids with very low melting point (35° C.), the CDs decorated with unsaturated with unsaturated aliphatic chains generates organogels with high viscosity at room temperature and thermally reversible viscosity allowing a facile control of the uptake and release upon change of the temperature. Organogels gained tremendous attention in the last years and often are based on long chain polymers. Modified CDs with aliphatic chains have never before been reported to be involved in the formation of molecular organogels. Crystallographic studies reported that all the materials are crystalline and the molecular adopt a lamellar structures indicative of the strong structural-directing proprieties of the aliphatic chains to form organized superstructures. These solid possess cavities to trap water soluble and insoluble guest molecules and can be released in specific medias. All the modified CDs are not soluble in water, contrary to the reported amphiphilic CDs derivatives. Embodiments of the present invention may utilize the supramolecular arrangement of the modified CDs to generate cavities associated either to the CDs and interstice space between the aliphatic chains to trap molecules in the solid state. These organogels may be dispersed in cosmetic formulation in order to release the active ingredients on controlled manner upon usage of the cosmetic and pharmaceutical products.

The previous studies may use amphiphilic CDs to design nanoparticles such water-soluble micelles and liposomes for the drug delivery. In embodiments of the present invention, the water insoluble amphiphilic CDs may be used as a building block for the formation of organogels or solids with very low melting point to host drugs or other active ingredients at room temperature and release the drugs or active ingredients upon the increase of the temperature.

Embodiments of the present invention include a porous organogel nanostructure that can host a wide variety of active ingredients, both water soluble and water insoluble. The OSMV are unique in that it can release the trapped ingredients when triggered by temperature and/or enzyme degradation. This invention is biodegradable cyclodextrin-based nanostructures that not only may stable in aqueous media but also may be eventually broken down for complete release of the encapsulated ingredients. Previously known structures either dissolve too easily due to higher water solubility of CDs (e.g., CD-MOF), do not breakdown readily (e.g., contain synthetic polymers) or are smaller nanoparticles forming micelles and liposomes made up of amphiphilic CDs. In contrast, OSMV can form large nanostructure agglomerates ranging in size from 100 nm to 500 um for higher ingredient storage capacity, stable in aqueous media, readily broken down by different enzymes, such as lipase enzyme found in the stomach, and may also has temperature-controlled release. The ability of OSMVs to remain stable in aqueous media but composed of molecules that ae readily broken down in these circumstances, e.g., cyclodextrins and fatty acids, may be advantageous to stabilize the active ingredients within the nanostructures and subsequently trigger API release in a controlled manner under external stimuli to provide a potent efficiency of the cosmetic products for long periods comparing to the already commercialized products. While the maximum API release rate in an API drug formulation may involve no encapsulation by OSMVs, the minimum rate may involve very large OSMV particle size such that it takes a very long time for the encapsulated ingredient to escape the OSMV network. Embodiments of the present invention include about 12-24 hour release for topical applications (demonstrated in FIG. 11b). For oral applications, the release rate may be slowed down further with other methods besides OSMV (e.g., pill coatings).

Embodiments of the present invention may chemically modify cyclodextrin with saturated and unsaturated fatty acids, such as Palmitoyl and oleoyl fatty acids, to form Organic Super Molecular Vessels (OMSV). The fatty acids may be attached to the CDs with ester functional group, of which hydrolysis may lead to the release of oleic acid and palmitic acid. These fatty acids may be biocompatible, biodegradable, and may provide numerous benefits for the skin. The OSMV based on oleoyl and palmitoyl derivatives can form respectively semicrystalline organogels and crystalline powders because of the favorable intermolecular interactions between the aliphatic chains. In addition, these molecular nanostructures may stack along one direction to form supramolecular nanotubes which can host small or long linear drugs. The ingredients may be loaded by mixing in a solvent and submerging the empty OSMV in the corresponding saturated solution. The ingredients diffuse into the OSMV and then the solvent can be filtered or evaporated. The incorporation of medicinal active ingredients within the cavity of the OSMVs can be achieved via a slow evaporation of organic solvents, while the release may be triggered slowly once the composites are in contact with aqueous media or when applied to the skin. In addition, the OSMV provide better thermal chemical stability of the medicinal active ingredients. Plain CDs may not necessarily be porous nanostructures, whereas CD-MOFs may comprise CDs arranged in a nanostructure. CD-MOFs may dissolve quickly in water and release the ingredients, whereas OSMV may be semi-stable in water and may “unwind” with temperature and also may be broken down by enzymes (skin, stomach, blood, and liver, etc. . . . ) to slowly break apart and thus release its encapsulated ingredients.

FIG. 1e depicts an Optimized molecular structure cyclodextrin modified with fatty acid chains in accordance with the principles of the present invention.

The OSMV consists of cyclic carbohydrates-based modified with saturated and non-saturated fatty acids such as palmitoyl and oleoyl fatty acids. Cyclic Carbohydrates such as α-, β-, and γ-cyclodextrin macrocycles are unique candidates for drug-carrier preparation since they possess several advantages, such as biocompatibility and biodegradability. The synthesis was achieved in high yield following a one-pot procedure using the palmitoyl and oleoyl chloride derivatives which react with the hydroxyl groups of the cyclodextrins (CDs). While saturated fatty acid leads to total esterification of the hydroxyl groups of the CDs, unsaturated aliphatic chains afford full substitution with the α- and β-CDs and partial substitutions with the γ-CD. The ¹H NMR titration determined that whilst the binding isotherm is a 2:1 host-guest model between the γ-CD and the niacinamide (NA) in aqueous media, the modified γ-CD with aliphatic chains bind NA in 1:1 host-guest isotherm in chloroform. The ester functional groups increase the affinity for hydrogen donor active ingredients while the hydrophobic nature of the CD cavity and the peripherical fatty acid chains increases the affinity towards hydrophobic active ingredients. Crystallographic studies revealed that the CD oleoyl esters (CDOs) are semicrystalline organogels and behaves like discotic liquid crystals, whilst the CD palmitoyl esters (CDPs) are crystalline powders at room temperature but melts at ˜35° C. Both CDOs and CDPs forms stacked superstructures with interlamellar distances in a range of 3.7-4 nm suggesting the formation of supramolecular nanotubes. The OSMV are soluble in organic solvents which allow loading medicinal active ingredients in organic solvents while the drug release will be achieved in aqueous media. The non-water-soluble nature of the OSMV plays a role of shield to slow down the solubility of the active ingredients in water and the cavity of the CDs can serve as a channel for hosting and releasing guest molecules. Study of the rheology of the γ-CDO, and β-CDO-1 revealed that possess strong gelation behavior at ambient condition while the viscosity decreases significantly at the biological temperatures (˜37° C.), a behavior which triggers the drug release. The temperature control of the self-assembly of oligosaccharide fatty acid esters which possess intrinsic cavity to host medicinal active ingredients offers numerous opportunities in the drug delivery technologies.

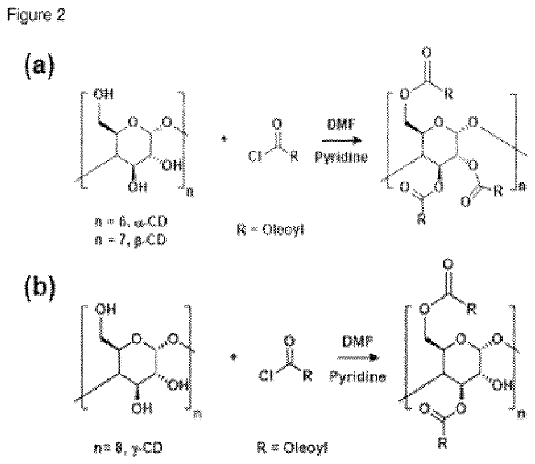

FIG. 2a depicts an illustration of the synthesis of α-CDO and β-CDO which possess 24 fatty acid chains in accordance with the principles of the present invention.

FIG. 2b depicts an illustration of the synthesis of γ-CDO which possess 16 fatty acid chains in accordance with the principles of the present invention.

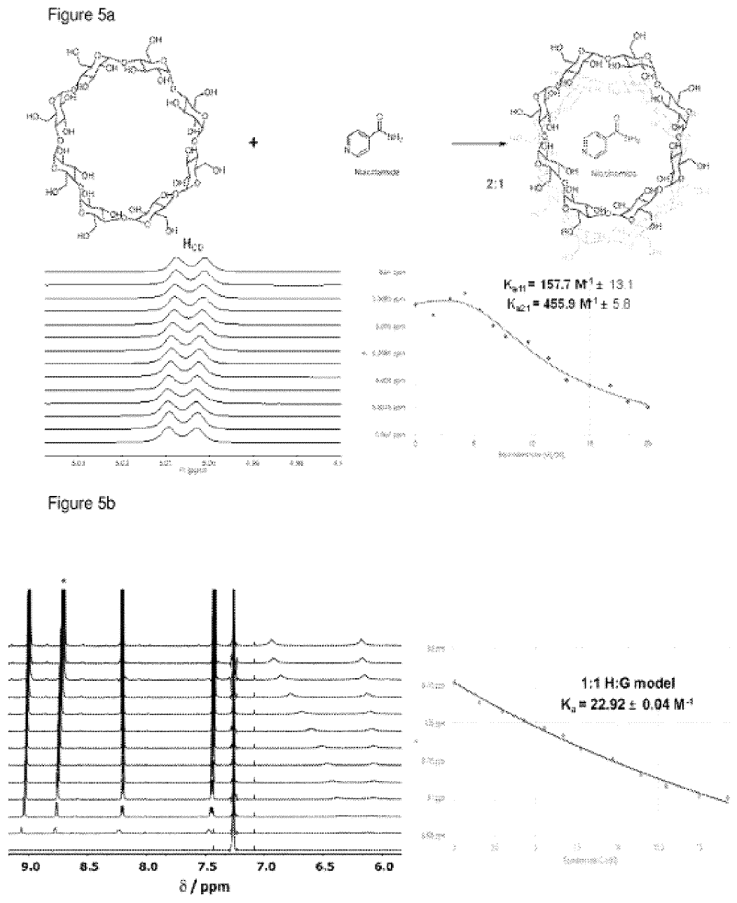

Embodiments of the present invention include a series of CDs (α, β, and γ-CDs) grafted with saturated (palmitoyl) and unsaturated (Oleoyl) fatty acid chains to generate supramolecular nanocapsules (CDPs and CDOs). Furthermore, these structures may host medicinal active ingredients (see FIGS. 2a, 2b, and 5a). The synthesis may be achieved in high yield using a one-pot synthesis process without using extensive and expensive purification procedures. While the CD palmitoyl ester (CDPs) may yield yellow powders, the unsaturated chains in the CD oleoyl esters (CDOs) may yield semicrystalline organogels. All the products have been analyzed by NMR and IR spectroscopies. In addition, ¹H NMR titration has been utilized to estimate the affinity the OSMVs towards medicinal ingredients such as niacinamide (NA). The crystalline nature of the assembly of the CDOs, CDPs and loaded CDO with NA in the solid state has been studies by powder XRD while the thermal stability of the OSMV were investigated using thermogravimetric analysis. The viscosity of the organogels have been studied at variable temperatures and determined that at the skin temperature. the viscosity may decrease significantly allowing a facile release of the medicinal active ingredients. This behavior may keep the integrity of the supramolecular assemblies of the drugs and the hosts at ambient temperatures while the release may be triggered when the temperature increases during usage.

### Synthesis and Characterization of the Cyclic Oligosaccharide Poly-Fatty Ester

The esterification of the cyclodextrins has been achieved by reacting the primary and/or secondary hydroxyl functional groups of the cyclodextrins with anhydrides, carboxylic acids, isocyanate, amides, and the purification is complex increasing therefore the production cost. The synthesis of the organic soluble substituted CDs with saturated (palmitoyl, α, β, and γ-CDP) and unsaturated (Oleoyl, α, β, and γ-CDO) polyesters followed a one-pot strategy in dimethylformamide (DMF) and pyridine under inert conditions (see FIGS. 2a and 2b). The palmitoyl chloride and oleoyl chloride may be exothermically reactive and upon their dropwise addition to the CDs already solubilized in DMF and pyridine may lead to an increase in the temperature of the solution. The color of the mixture may turn pale yellow/orange. The pyridine may play a crucial role to avoid the decrease of the pH of the solution as a result of the release of hydrochloric acid (HCl) as a byproduct. At acidic pH, the CDs may decompose by cleaving the α-1,4-linked d-glucopyranosyl bond. The CDOs and CDPs may be insoluble in water and therefore were purified by water/organic extraction, which removes the water soluble reactive and byproducts such as cyclodextrin, pyridium chloride and DMF. In addition, a final wash of the CDOs and CDPs using ethanol leads to the removal of other byproducts such as oleic and palmitic acid because of the non-soluble nature of the compound of interest in alcohols. Fatty acids may have relatively weak affinity for CDs which facilitate their elimination in ethanol. All the compounds of the present invention have been isolated in high yield (>95%) offering therefore a possibility to scale up the reaction and reduce the cost of the production. The CDPs may comprise solids with melting point temperatures ranging from 35-40° C., while the CDOs may comprise highly viscous organogels.

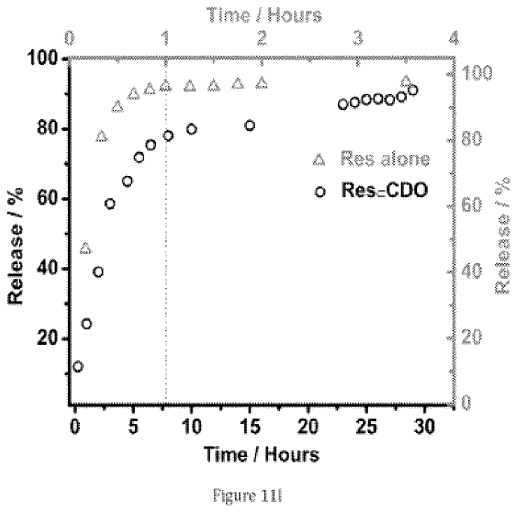

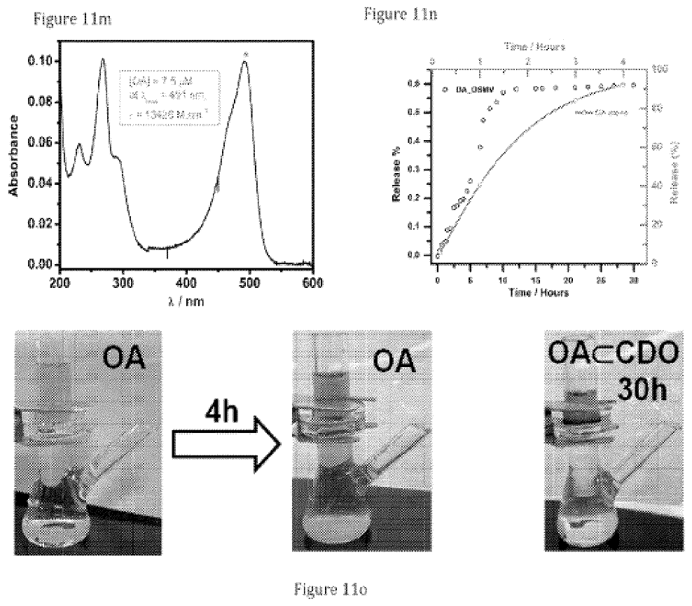

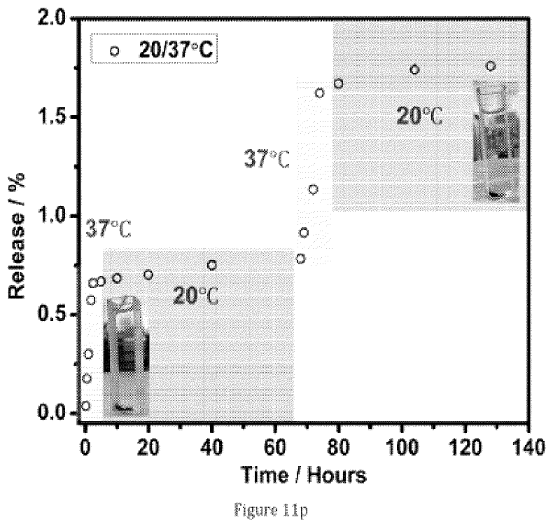

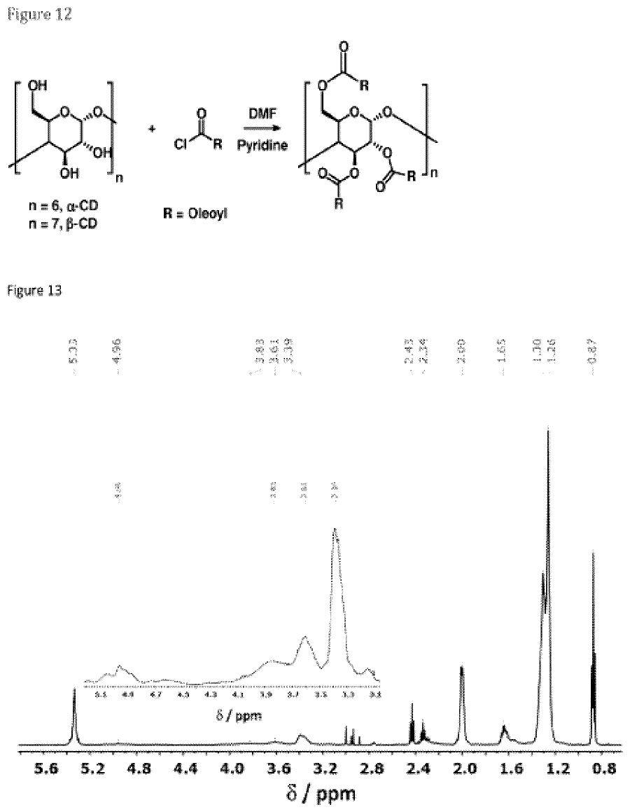

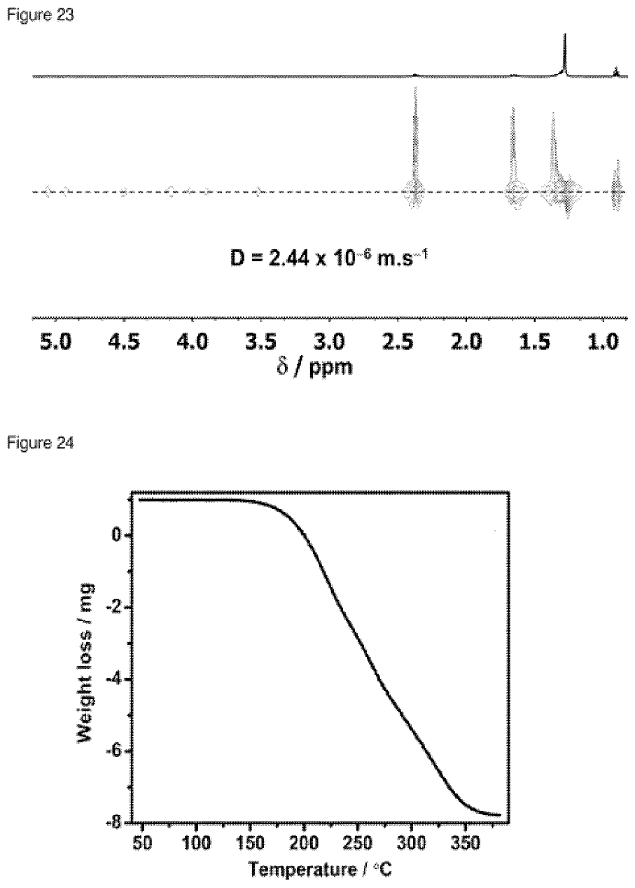

The ¹H NMR spectroscopy revealed that all the spectra of the CDOs and CDPs display broad peaks in a region 3-5 ppm characteristic of the proton resonance of the carbohydrate moiety (See FIGS. 12-26 and corresponding description). The integration of these peaks offered an estimation of 18, 21, 16 of oleoyl chains attached to one molecule of α, β, and γ-CDs respectively. This may indicate that the substitution occurs on both sides of the cyclodextrin on both the primary and secondary hydroxyl groups. In some embodiments, the α and β-CD may be fully substituted with oleoyl aliphatic chain. However, in some embodiments, the γ-CD may be partially substituted. ¹H NMR of the CDPs revealed that higher ratios of palmitoyl chains may be attached to the CDs with an estimation of numbers of chains of 18, 21 and 24 aliphatic chains attached to respectively the α, β, and γ-CDs. ¹H NMR Diffusion Ordered Spectroscopy (DOSY) experiments conducted on γ-CDO (FIG. 17) and γ-CDP (FIG. 23) revealed the absence of small molecules such as free fatty acids or lower substituted CDs. In addition, the coefficient of diffusion of γ-CDO is of 2.19×10⁻⁶ m·s⁻¹ slightly small than this of γ-CDP, 2.44×10⁻⁶ m·s⁻¹ indicative of the similarity of molecular weight and molecular volume of the γ-CDO and γ-CDP. These coefficients from Diffusion NMR may resolve size of molecules or aggregates and degree of polymerization. The similar coefficients may mean that the OSMV produced from oleolic acid ester and palmitic acid ester may be similar. However, since the corresponding OSMVs may be similar in weight and volume, they may comprise similar cavities and so trapped loaded active ingredients may have similar outgoing diffusion rates.

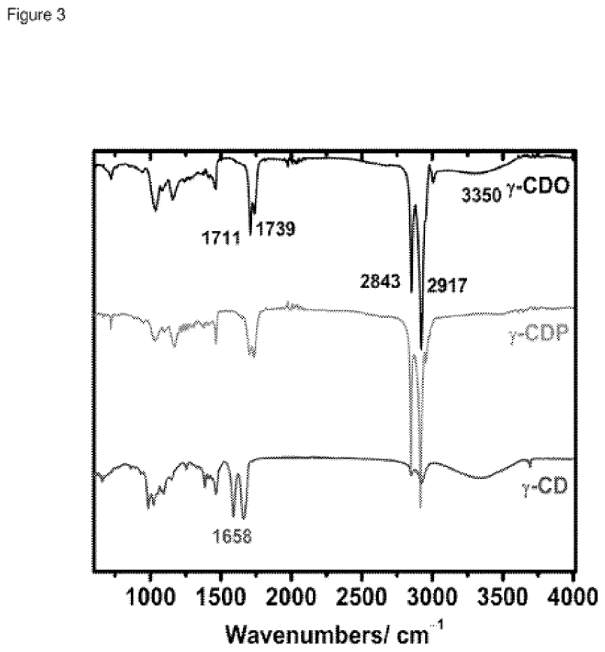

FIG. 3 depicts the FT-IR spectra of γ-CD, γ-CDP and γ-CDO in accordance with the principles of the present invention.

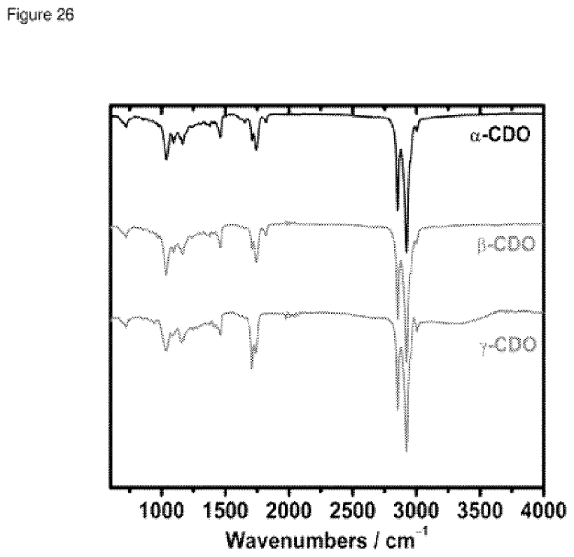

In the IR spectra of γ-CD and γ-CDO (FIG. 3 and FIG. 26) a wide band was observed with an absorption maximum at 3350 cm⁻¹. This may be a result of the valence vibrations of either the O—H bonds of the primary and secondary hydroxyl groups indicative of the partial esterification of the CDs. The IR spectra of the CDPs do not necessarily display this wide band indicative of complete esterification of the both the primary and the secondary hydroxyl groups of the CDs. An absorption band may also be observed, belonging to the valence vibrations of the C—H bonds in the CH and CH₂ groups with a maximum at 2917 cm⁻¹. The absorption bands from the deformation vibrations of the C—H bonds in the primary and secondary hydroxyl groups are observed in the region 1400-1200 cm⁻¹. The bands from the valence vibrations of the C—O bonds in the ether and hydroxyl groups of the CDs are observed in the region 1200-1000 cm⁻¹. The γ-CDO display and sharp peak at 3011 cm⁻¹ associated to the alkene functional group of the oleoyl aliphatic chain indicative of the persistence of the chemical integrity of the aliphatic chain in the reaction conditions and purification process. Whilst the IR spectrum of the γ-CD display peaks at 1595 and 1658 cm⁻¹ which reflect the δ-HOH bending of water molecules attached to CDs, the γ-CDP and γ-CDOs show peaks at 1711 and 1739 cm⁻¹ were assigned to the C═O stretching of the ester functional groups.

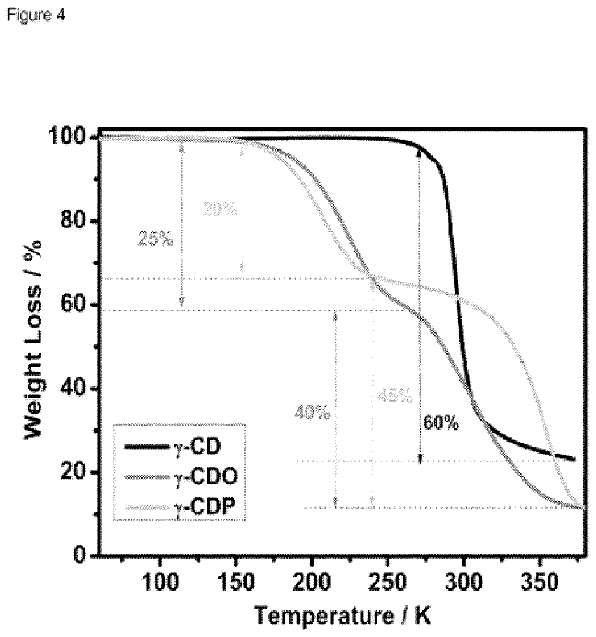

FIG. 4 depicts a thermogravimetric analysis of the γ-CD, γ-CDO and γ-CDP in accordance with the principles of the present invention.

The thermal stability of the γ-CD. γ-CDP and γ-CDP has been investigated (FIG. 4) using thermogravimetric analysis which was carried out in argon atmosphere. The γ-CD display in a temperature range, mostly 250-380° C., which is associated with a weight loss of 60%, with the formation of a residue associated to the decomposition of the CD. Remarkably, the γ-CDO and γ-CDP have a different thermal profile showing respectively a first weight loss of 20 and 25% at the temperature range of 190-260° C. This weight loss may be associated to the decomposition of the ester functional groups attaching the aliphatic chains to the CD. A second weight loss occurs at the temperature range of 260-385K corresponding to the decomposition of the CD. Substitution of the OH groups may lead to noticeable effect of the structure of the cyclodextrins on their thermal stability. For example. the tosyl group may decrease the degradation temperature of the β-CD from 314 to 187° C. The high thermal stability of the CDOs and CDPs can provide additional stabilization of medicinal guest molecules. For instance, CDs encapsulating eugenol presents enhanced thermal stability, and slow release at high temperatures.

FIG. 5a depicts a ¹H NMR Spectra recorded in H₂O show changes in chemical shifts of γ-CD upon increasing the concentration of NA in accordance with the principles of the present invention.

FIG. 5b depicts a ¹H NMR Spectra of the γ-CDO and NA recorded in CDCl₃ show changes in chemical shifts upon increasing the concentration of NA in accordance with the principles of the present invention.

Complexation of Niacinamide with the OSMV

With OSMVs, whether the presence of the aliphatic chains have any effect on its host-guest chemistry in comparison with pristine CDs was determined. A series of ¹H NMR titrations (FIG. 5b) in which incremental additions of solutions of niacinamide (NA) in CDCl₃ were made to a solution of a host. The addition of the NA to a solution of γ-CDO resulted in significant shifts in the δ values of selected protons on NA—as expected for complexation—and the data were fitted to a 1:1 binding isotherm. The binding affinities (expressed as Ka values) of γ-CDO toward NA was found to be 22.9±0.04 M⁻¹. The resonance of the NH₂ protons of the NA shift significant down field as the result of the existence of hydrogen bounding with the ester functional groups. The binding isotherm of the NA and γ-CDs in H₂O was determined to be a 2:1 Host:Guest model with binding affinities of 157 and 455 M¹ (FIG. 5a). The 2:1 model can be favored over a 1:1 model because the guest molecules may mediate the hydrogen bonding between CD molecules. In addition, the large affinities of the NA towards γ-CDs can be associated to the existence of hydrogen bonding between the amide and hydroxyl functional group of the NA and CD respectively. At higher substrate concentrations, the resonance NH₂ protons may split into two resonances reflecting the inequivalent chemical environment of the two protons upon complexation of NA with γ-CDO through hydrogen bonding with the ester functional groups. Having determined the effect of incorporating the fatty acid chains on the binding of guests in the solution phase, attention was turned to investigating the complexation behavior of OSMVs toward NA and other active medicinal ingredients in the solid state.

Studies of the Dynamic Viscosity

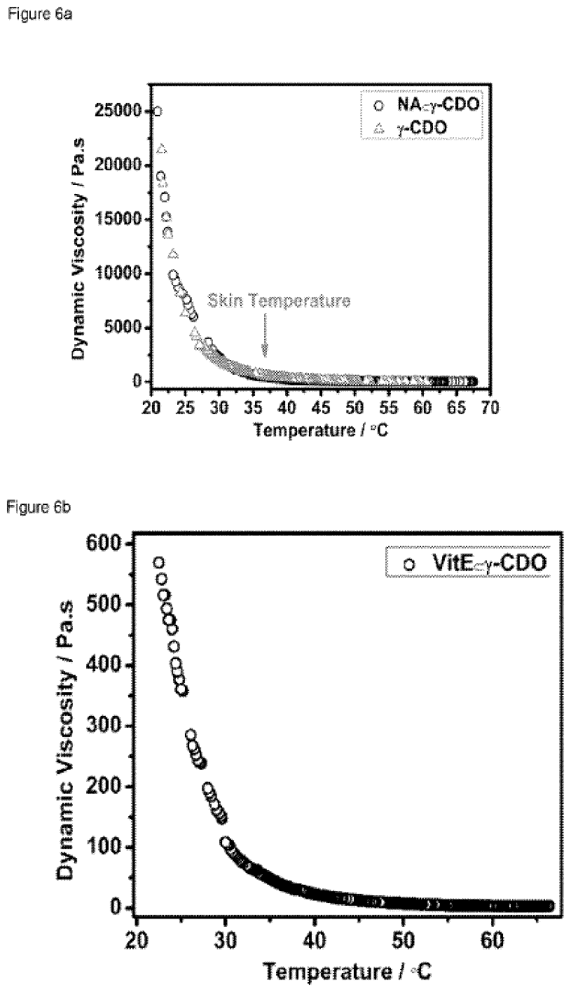

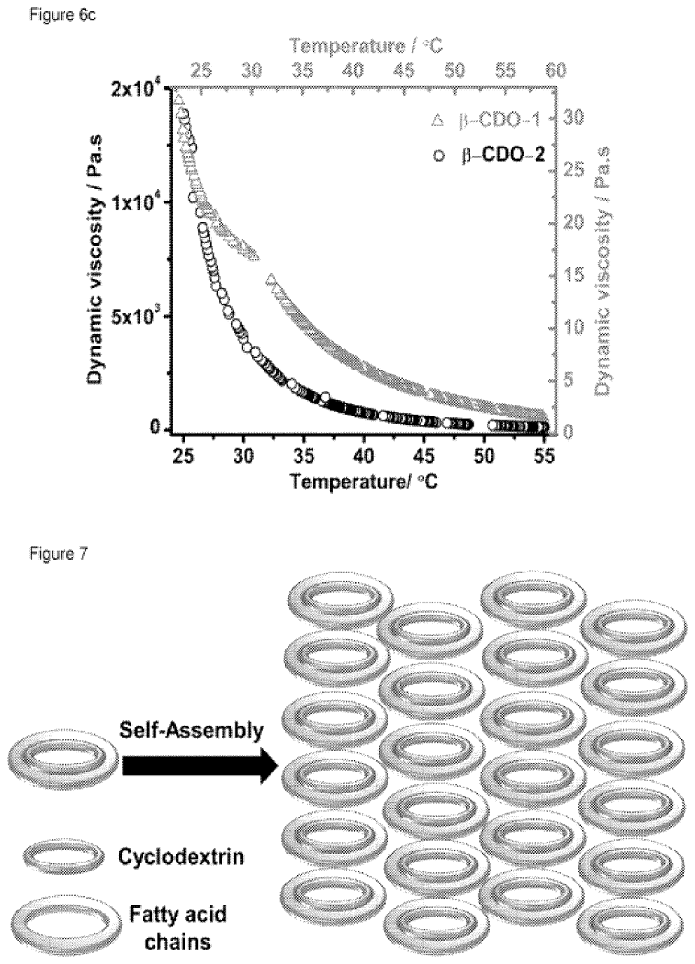

The dynamic viscosity data as a function of temperature were collected for γ-CDO, NA⊂γ-CDO, and α-tocopherol⊂γ-CDO and displayed in FIG. 6a-c. The dynamic viscosity decreases exponentially with increasing temperature as a result of the weakening of the intermolecular interactions. The dynamic viscosity of the γ-CDO at 21.5° C. is 21.46 kPa·s, while at 60.5° C. the viscosity is 720 times less, reaching 0.0295 kPa·s. The viscosity of oleic acid was reported to be 0.0348 Pa·s at 20° C., while this of the γ-CDO is of 21.46 kPa·s at 21.5° C. indicative of the cooperative effect of the aliphatic chains attached to the CDs in increasing the gelation process.

FIG. 6a depicts a graph of the dynamic viscosity of the γ-CDO and NA⊂γ-CDO as function of temperature in accordance with the principles of the present invention.

The formation of molecular gels may result from molecular assembly. Initially, driven by supersaturation, nucleation may occur between gelator molecules. The nucleating centers form one-dimensional (1D) objects usually, such as fibers, rods, ribbons, tapes, platelets and tubules. The interactions that promote preferential 1D growth may include electrostatic interactions, packing constraints, H-bonding, π-π stacking, dipolar interactions, hydrophobicity or hydrophilicity, and London dispersion forces. The 1D objects may bundle into objects with larger cross-sections and may interact further to form the three-dimensional (3D) assembled networks that immobilize the liquid. Organogels have been investigated because of their numerous potential applications as controlled drug delivery devices. In this context, preparation of biocompatible organogels that possess a cavity to host medicinal active ingredients can find application in pharmaceutical and cosmetic industry. Fatty acid esters with short aliphatic chains (<26 carbons) do not necessarily form gels at room temperature due to the weak intermolecular interactions. In longer chains (>26 carbons), the London dispersion forces may become dominant and the gelation can occur. Also, fatty acids may have better tendency for gelation as a result of the H-bonding interactions between carboxylic acid functional groups.

The dynamic viscosity data as a function of temperature (15-53° C.) were collected for γ-CDO and displayed in FIG. 6. The dynamic viscosity decreases exponentially with increasing temperature as the result of the weakening of the intermolecular interactions. The viscosity of the γ-CDO at 14.8° C. is 230.2 Pa·s while may be 50 times less reaching 4.5 Pa·s at 53° C. The viscosity of oleic acid was 0.0348 Pa·s at 20° C., while this of the γ-CDO is of 78.4 Pa·s at the same temperature may be indicative of the cooperative effect of the aliphatic chains attached to the CDs to increase the gelation process. Crystallographic studies revealed that γ-CDO may be semicrystalline and may be indicative of the formation of well-ordered superstructures. The dynamic viscosity of the NA⊂γ-CDO at variable temperatures reproduced similar behavior of the γ-CDO alone indicative that the physical properties of the composite are essentially dominated by the assembly of the γ-CDO and the persistence of the interaction between the fatty acid chains. In addition, the gelation process is fully reversible. Such behavior may increase the stability of the encapsulated active ingredients at low temperature and the release can be triggered upon the decrease of the viscosity at higher temperatures. Typical skin temperature may be about 33° C. and the viscosity of γ-CDO at similar temperature may be 14.6 Pa·s an order of magnitude higher than this at 17° C. Thus, porous organogels may be beneficial to design new porous organogels with integrated high performances in drug delivery for skin care products.

FIG. 6b depicts a graph of the dynamic viscosity of α-tocopherol⊂γ-CDO as function of temperature in accordance with the principles of the present invention.

FIG. 6c depicts a graph of the dynamic viscosity of the β-CDO-1 and β-CDO-2 as function of temperature in accordance with the principles of the present invention.

Crystallographic studies revealed that γ-CDO is semicrystalline indicative of the formation of well-ordered superstructures. The dynamic viscosity of the NA⊂γ-CDO and α-tocopherol⊂γ-CDO at variable temperatures reproduced (FIGS. 6a-b) similar behavior of the γ-CDO with a fast gelation below 30° C. indicative that the physical properties of the composite are essentially dominated by the self-assembly of the γ-CDO and the persistence of the interaction between the fatty acid chains. It is noteworthy that NA⊂γ-CDO and α-tocopherol⊂γ-CDO composites have been prepared using a mole ratio of 6:1 and 3:1 of the NA: γ-CDO and α-tocopherol: γ-CDO respectively. In this context, the low viscosity of the α-tocopherol⊂γ-CDO (569 Pa·s at 22.5° C.) compared to γ-CDO (13626 Pa·s at 22.5° C.) might be associated to the unloaded α-tocopherol which possess a low viscosity (4.6 Pa·s) at 20° C. Interestingly. the gelation process is fully reversible. Such behavior is important to increase the stability of the encapsulated active ingredients at low temperature and the release can be triggered upon the decrease of the viscosity at higher temperatures. Indeed, the skin temperature is reported to be of 33-37° C. and the viscosity of γ-CDO at this temperature range is 0.56-1.2 kPa·s, an order of magnitude lower than the dynamic viscosity at 21.5° C. (21.46 kPa·s).

To confirm that the gelation of the CDOs is exclusively associated to the fatty acid chains, we prepared two β-CDO derivatives with different number of fatty acids attached to the β-CD. In the β-CDO-1, all the hydroxyl group of the β-CD have been substituted with oleic acid chains, while in the β-CDO-2 only 14 mole equivalents of oleoyl chains have been attached to the β-CD. The viscosity of β-CDO-1 at 25° C. is 20.9 Pa·s while this of β-CDO-2 is more than 600 times higher reaching 13.8 kPa·s at the same temperature (FIG. 6c). In addition, at variable temperatures, the viscosity ratios between the high (55° C.) and low (25° C.) temperatures for β-CDO-1 and β-CDO-2 are respectively of 14 and 107 indicating the stronger tendency of the β-CDO-2 for gelation compared to β-CDO-1. The discrepancy in the gelation behavior of β-CDO-1 and β-CDO-2 confirms the existence of a fine-balance between the number of chains attached to the CD and the viscosity of the gel. It is worth noting that both the β-CDO-1 and γ-CDO have respectively 14 and 16 chains attached, and the viscosity is significantly higher compared to the fully substituted CDs. We believe this work provides a new insight for porous organogels, which is beneficial to design new porous organogels with integrated high performances in drug delivery for skin care products.

### Crystallographic Studies:

FIG. 7 depicts a pictogram representation of columnar discotic superstructures in accordance with the principles of the present invention.

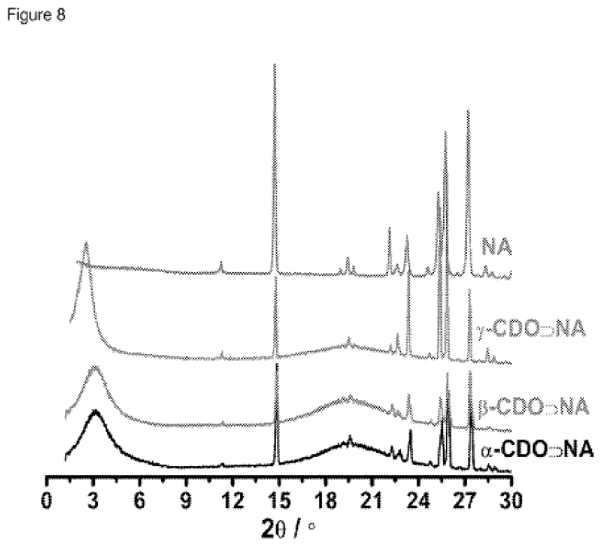

FIG. 8 depicts the powder X-ray diffraction patterns of the NA, NA⊂γ-CDO, NA⊂β-CDO and NA⊂γ-CDO in accordance with the principles of the present invention.

The molecular arrangements of the CDOs and CDPs has been examined by powder X-ray diffraction. The powder diffraction pattern of the CDOs display two diffraction peaks at low and at high angle indicative of the semicrystalline nature of the Gels. In γ-CDO, the three Bragg diffraction peaks at 2.53° and 19.5°, corresponding to the spacings of 3.75 and 0.45 nm, respectively. The spacing of 0.45 nm was assigned to the lateral packing of oleoyl aliphatic chains, suggesting that the alkyl chains adopt a linear conformation. This result was consistent with the JR spectra. The first peak 2q=2.53° is attributed to (001) Bragg peaks of a lamellar structure with a layer distance of 3.75 nm. This behavior may be similar to the behavior of discotic liquid crystals where molecules may adopt columnar superstructures (FIG. 7). The α and β-CDOs display diffraction pattern with low angle diffraction peak at 2q=3.12° and a broad peak at 2q=19.5°. The similarity of the diffraction patterns between the α, β, and γ-CDOs indicates the similarity in the overall supramolecular packing, although the interlamellar distances vary slightly.

The crystallization of the CDOs with niacinamide (NA) at a molar ratio of 1:6 CDO:NA was achieved by slow evaporation of ethyl acetate (EtOAc). Niacinamide may be useful for topical cosmetic application. This loading ratio may be applicable for slower release as it comes out to about 11% loading ratio. It is noteworthy that NA has a low solubility in EtOAc, however upon addition of the CDOs, its solubility increases significantly confirming the relatively favorable affinity of the NA to the cavity of the CDOs as determined by ¹H NMR titration. The powder pattern of the mixture revealed (FIG. 8) that two phases exist arising from the crystalline phase of the NA and CDO. Hence, the inclusion of the guest molecules inside the cavity of the CDOs do not disturb the arrangement of the CDOs as a result of the favorable interactions between the fatty acid chains. These results are consistent with the viscosity data showing the similar behavior of the γ-CDO and NA⊂γ-CDO.

The modified CDs with saturated fatty acids, α, β, and γ-CDP may form solids powders at room temperature but melts at ˜35° C. Similarly, to the CDOs, which display a significant change in the viscosity at different temperature, the low melting point of the CDPs, can be beneficial to trigger drug delivery upon the increase in temperature. Skin temperature may be about 33 C. This temperature may significantly increase the viscosity of OSMV to “unwind” and therefore release the encapsulated ingredients. The rate can be controlled and tuned for about 12 hours for 100% cumulative delivery.

FIG. 8(top) depicts the X-ray diffraction patterns of α, β, and γ-CDP at room temperature. The β and γ-CDPs show a strong diffraction peak in the small-angle region (2θ=2.47° and 2.46° respectively) associated to the Bragg diffraction peaks (001), while in the α-CDP, the peak is broad with maximum intensity at 20=2.77°. All the compounds crystallize in the monoclinic space group with two long axes (˜37 Å) while the Q-axis is shorter (˜3.5 Å). The CDPs forms columnar structures similar to discotic liquid crystals (FIG. 7) undoubtedly due to the favorable interactions between the fatty acid chains. As expected, the cell volume may increase with the increase of the size of the CD. Previous studies reported a series of amphiphilic β-CD derivatives capable of forming hydrogen bonding networks of different strengths have been synthesized to probe the role of the hydrogen bonding network in the formation of assembled mesophases.

TABLE 1
Cell parameter of the α, β and γ-CDP

| | | |
|---|---|---|
| α-CDP | β-CDP | γ-CDP |

| h k l | 2θ/° | h k l | 2θ/° | h k l | 2θ/° |
|---|---|---|---|---|---|
| 1 0 0 | 2.776 | 1 0 0 | 2.472 | 1 0 0 | 2.46 |
| 0 0 1 | 3.017 | 0 0 1 | 3.165 | 0 0 1 | 2.975 |
| 0 0 2 | 6.032 | 1 0 −2 | 6.266 | 2 0 0 | 4.912 |
| 2 0 2 | 8.322 | 1 0 2 | 7.389 | 2 0 −2 | 7.366 |

| | a/Å | b/Å | c/Å | β/° | V/Å³ |
|---|---|---|---|---|---|
| α-CDP | 31.8456 | 3.41 | 29.2692 | 91.36 | 3177.59 |
| β-CDP | 36.9616 | 3.458 | 28.6207 | 104.137 | 3547.35 |
| γ-CDP | 36.0683 | 3.6974 | 29.868 | 94.978 | 3968.09 |

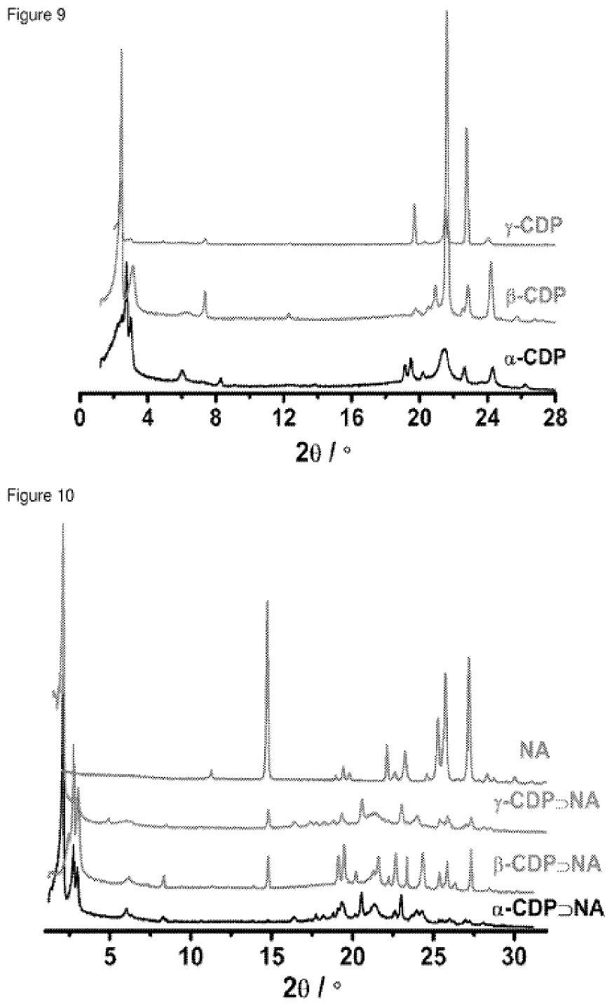

FIG. 9 depicts the powder X-ray diffraction patterns of the α-CDO, β-CDP and γ-CDP in accordance with the principles of the present invention.

FIG. 10 depicts the powder X-ray diffraction patterns of the NA, NA⊂γ-CDO, NA⊂β-CDP and NA⊂γ-CDP in accordance with the principles of the present invention.

The cocrystalisation of the CDPs with NA offered (FIG. 10) similar diffraction pattern although they present subtle differences. The (100) and (001) Bragg diffraction peaks are slighted shifted to be at 20=2.09° when NA is incorporated into the cavity of the α and γ-CDPs while the β-CDP remain unchanged. The decrease in the 2q may indicate that the interlamellar distances between the CDOs may be increasing due to the incorporation of the NA inside the cavity.

### Drug Release in Aqueous Media:

Preliminary drug release studies have been conducted on the Resveratrol⊂γ-CDO (5 mg) host-guest complex in deionized H₂O (3 ml) using UV-visible spectroscopy. The resveratrol has a strong absorption band are 315 nm (FIG. 11a) facilitating monitoring the concentration of the resevertarol released to the solution. Noteworthy upon addition of water to the Resveratrol⊂γ-CDO complex, the intensity of the absorbance may be small (0.46) and the intensity may increase slightly after 100 min (FIG. 11b), which may indicate the ability of the OSMV to enhance the retention of the medicinal active ingredients in aqueous media. After 100 min, the resveratrol release may be accelerated and the maximum absorbance may reach 4.8 at 700 min. The one fold increase of the reseveratrol concentration in water after 12 hours, confirm the ability of the cavity of the OSMV superstruture to host and release medicinal active ingredients.

Gel delivery systems can leverage therapeutically beneficial outcomes of drug delivery and can provide spatial and temporal control over the release of various therapeutic agents, including small macromolecular drugs. The gelator molecules immobilise large volumes of liquid following their self-assembly into a variety of aggregates such as rods, tubules, fibres and platelets. Owing to their thermoreversibility, controllable degradability and capability to protect labile drugs from degradation, hydrogels or organogels serve as a platform in which various physiochemical interactions with the encapsulated drugs control their release. Only a few organogels, however, are currently being studied as drug delivery vehicles, as most of the existing organogels are composed of pharmaceutically unacceptable organic liquids and/or unacceptable/untested gelators. In this context, the CDOs gels based on natural ingredients (fatty acids and carbohydrates) can find application as drug delivery system owing to not only their gelation properties but also to the CD cavity which from supramolecular channels that can host drug molecules.

FIG. 11(a) depicts the kinetics of resveratrol release from the Resveratrol⊂γ-CDO complex in accordance with the principles of the present invention.

FIG. 11(b) depicts the UV-visible absorption spectra collected at room temperature plot of the intensity of absorbance at 315 nm as a function of time in accordance with the principles of the present invention.

Several Organic Super Molecular Vessels (OSMVs) based on decorated cyclodextrins (CDs) with fatty acid chains have been prepared following a one-pot synthesis using CDs and acyl chlorides. While the cavity sizes of the a, s, and γ-CDs may allow fine-tuning the affinity of the host to the guest molecules, the fatty acid chains play a structure-directing role to form superstructures with desirable physical properties. Whilst CDs decorated with saturated fatty acids such as palmitoyl fatty chains (Cyclodextrin palmitoyl esters, CDPs) offers crystalline powders with low melting point (˜35° C.), the CDs modified with unsaturated fatty chains such as oleoyl units (Cyclodextrins oleoyl esters, CDOs) offer organogels with high viscosity at room temperature but the viscosity decreases significantly at biological temperatures offering therefore a potential application in drug delivery technologies. Powder X-ray diffraction revealed that both the CDPs and CDOs adopts a stacked superstructure with interlamellar distance of ˜3.7 nm which increases to 4 nm upon incorporation of guest molecules inside the cavities. Incorporation of active ingredients within the OSMV may increase their stability and prevent side reactions between ingredients. In addition, the OSMVs may be biocompatible and can find application in both cosmetic and pharmaceutical industry.

FIG. 11c depicts a graph of the kinetic of the resveratrol release from the Resveratrol⊂γ-CDO composite in ethanol and water monitored using UV-visible spectroscopy at 315 nm in accordance with the principles of the present invention.

FIG. 11d depicts a graph of the kintic of the release of α-tocopherol from α-tocopherol⊂γ-CDO composite monitored using UV-visible spectroscopy at 295 nm in accordance with the principles of the present invention.

Preliminary drug release studies have been conducted on the Resveratrol⊂γ-CDO (5 mg) host-guest complex in deionized H₂O (3 ml) using UV-visible spectroscopy. The resveratrol has a strong absorption band at 315 nm (FIG. 11c) facilitating monitoring the concentration of the resevertarol released to the solution. Noteworthy, upon addition of water to the Resveratrol⊂γ-CDO complex, the intensity of the absorbance is very small (0.46) and the intensity increases slightly after 100 min (FIG. 11d) indicating the ability of the OSMV to enhance the retention of the medicinal active ingredients in aqueous media. After 100 min, the resveratrol release is accelerated and the maximum absorbance reaches 4.8 at 700 min. The one fold increase of the reseveratrol concentration in water after 12 hours, confirm the ability of the γ-CDO to enhance its retention to prevent its solubilisation in water. The solubility of reseveratrol in water is relatively poor (0.03 mg/ml) which can hamper a fast release of the resveratrol. Resveratrol is freely soluble in ethanol (50 mg/ml), while the γ-CDO is unsoluble in EtOH which allows the study of the release water insoluble medicinal ingredients in organic medium. FIG. 11c shows that while the overall release percentage of resveratrol in water do not exceed 1.5% after 12 hours, the release in EtOH is fast during the first 2 hours reaching 14%, then it slows down to an cumulative release of 22% after 12 hours. In the case of magnesium ascorbyl phosphate (MgAsc), is slightly more soluble in water (8.1 mg/ml) and the release kinetic profile of MgAsc⊂γ-CDO reveals (FIG. 11d) once gain a fast release during 3 hours then the release stops to reach 22% cumulative release after 12 hours.

FIG. 11d depicts a graph of the release kinetic of the magnesium ascorbyl phosphate (MgAsc) from the MgAsc⊂γ-CDO composite in deionized water. The concentration of MgAsc was monitored by UV-vis absorption spectroscopy at 260 nm in accordance with the principles of the present invention.

These results indicate that the solubility of the guest medicinal molecules do influence the release kinetic, although the cumulative release remains low, supporting that the stability of the loaded ingredients within the γ-CDO matrix at room temperature in highly solubilising media.

The retention of active ingredients within the γ-CDO have been tested for other medicinal ingredients which are more soluble in water. The niacinamide is highly soluble in water with a solubility of 1 g/ml while resveratrol has a solubility of 0.05 mg/ml. The γ-CDO and Niacinamide (NA) have been determined by ¹H NMR titration to form a 1:1 host-guest complexes in solution. In order to enhance the NA loading into γ-CDO, a 1:6 mole ratio of NA:γ-CDO has been utilized and the crystallization process was achieved through a slow evaporation of organic solvent. The crystallographic studies have revealed that the supramolecular arrangement of the γ-CDO is not disturbed when co-crystallized with small molecules that can be hosted inside the supramolecular channels. In contrast, the drug release process was achieved in aqueous media where the water insoluble γ-CDO plays a role of a molecular shield to protect the NA from being solubilized in water. The release studies were achieved using the Franz diffusion cell at 23 and 37° C. in order to test the effect of temperature on the drug release kinetic. The viscosity experiments revealed a significant change in the rheology within a temperature range of 20-37° C. For the experiments at 23 and 37° C., 55 mg and 26 mg of the NA⊂γ-CDO was deposited on the artificial membrane, then placed in the Franz diffusion cell. The cell was filled with 15 ml deionized water and 3 ml water are utilized as mobile eluent. At selected time intervals, 3 ml of aqueous solution was withdrawn from the release medium and the NA concentration in solution was monitored by absorption spectroscopy. The sample was put back to the release medium after the measurement

FIG. 11(e) depicts a graph of the release kinetic of the magnesium ascorbyl phosphate (MgAsc) from the MgAsc⊂γ-CDO composite in deionized water. The concentration of MgAsc was monitored by UV-vis absorption spectroscopy at 260 nm in accordance with the principles of the present invention.

FIG. 11(f) depicts a graph of diffusion of the Niacinamide from the γ-CDO using the Franz diffusion cell where the concentration of Niacinamide is monitored by UV-Visible spectroscopy experiment was conducted at 23° C. in accordance with the principles of the present invention.

FIG. 11(g) depicts a graph of diffusion of the Niacinamide from the γ-CDO using the Franz diffusion cell where the concentration of Niacinamide is monitored by UV-Visible spectroscopy experiment was conducted at 37° C. in accordance with the principles of the present invention.

FIG. 11(h) depicts a graph of the diffusion kinetics of niacinamide (NA) from the NA⊂γ-CDO complex using Franz diffusion cell at 23 (lower trend) and 37° C. (upper trend) in H₂O in accordance with the principles of the present invention. The concentration of the NA was monitored by absorption spectroscopy at the wavelength of 260 nm.

FIG. 11(i) depicts a graph of the diffusion kinetics of niacinamide (NA) alone using Franz diffusion cell at 23 (lower trend) and 37° C. (upper trend) in H₂O in accordance with the principles of the present invention. The concentration of the NA was monitored by absorption spectroscopy at the wavelength of 260 nm.

The absorption spectrum of NA revealed (FIG. 11f-g) the presence of a strong absorption band at 261 nm (ε=8764 M·cm⁻¹). From the molar ratio of NA:γ-CDO of 6:1, the maximum concentration of the NA expected to be leached to the water solution can be estimated. For both experiments at 23 and 37° C., a maximum concentration of 0.662 and 0.311 mM respectively are expected and hence the percentage of the NA to be released experimentally can be monitored (FIG. 11h-i). During the first 2.5 hours, the release kinetics at 23 and 37° C. are almost identical reaching (FIG. 11-5h-i) a maximum release of 8 and 11% respectively. Interestingly at 23° C., the release reaches a plateau after 5 hours with a maximum NA release of 18% and increases to 22% after 48 hours although the high solubility of NA in water (1 g/ml).

FIG. 11(j) depicts a graph of the absorption spectra of the Ginseng, Gotu Kola and Green Tea water extract at concentration of 0.4 mg/L in accordance with the princinples of the present invention.

FIG. 11(k) depicts a graph of the kinetic of the drug release in deionized water in accordance with the princinples of the present invention.

To confirm the protective effect of the γ-CDO from the solubilization of medicinal ingredients in aqueous media, we loaded the γ-CDO with Green tea (GT), Ginseng (GS) and Gotu Kola (GK) water extract by forming emulsification phase followed by an evaporation of the organic and aqueous solvents under high vacuum. 300 mg of the formed composites are placed inside a UV-visible cuvettes then 4 ml deionized water was added. The kinetic of the diffusion of the active ingredients from the OSMV are monitored in solution using UV-visible spectroscopy at the λₘₐₓ=300 nm (FIG. 11-6j) at room temperature (23° C.). As expected, the release of the water-soluble ingredient was achieved gradually with a fast release during the initial 2 hours and then the leaching process of the optically active ingredients slowdown (FIG. 11-6k) after 4-5 hours.

These studies reflect that the fast leaching of ingredients during the initial four hours are associated to the unloaded ingredients which are present on the surface of the gel. Previous studies (Zhou, Z.; He, S.; Huang T.; Peng, C.; Zhou, H.; Liu, Q.; Zeng, W.; Liu, L.; Huang, H.; Xiang, L.; Yan, H. Preparation of gelatin/hyaluronic acid microspheres with different morphologies for drug delivery. Polymer Bulletin, 2015, 72, 713-723) reported the observation of similar behavior with burst a release related to some drugs being weakly bonded to the surface of the composites. The guest molecules present within the supramolecular channels and interstice of the γ-CDO superstructures are characterized by a superior retention and protection from the aqueous media at ambient temperatures on account of the rigid nature of the water repellant γ-CDO gel.

FIG. 11(l) depicts a graph of the diffusion kinetics of resveratrol using Franz diffusion cell at 37° C. in EtOH. The concentration of the resveratrol was monitored by absorption spectroscopy at the wavelength of 315 nm. Diffusion of resveratrol from the resveratrol⊂CDO complex (lower line), and diffusion of the resveratrol (upper line) in accordance with the principles of the present invention.

Remarkably, the experiment at 37° C. have shown (FIGS. 11(h)-(i)) a NA release of 31% after 5 hours and the release continues gradually to reach 94% after 46 hours. Other experiments with resveratrol⊂γ-CDO using the Franz diffusion cell at 37° C. in EtOH revealed (FIG. 11(l)) a cumulative drug release of 80% after 10 hours and reaches 91% resveratrol after 29 hours. These results are consistent with the viscosity experiments showing a two order of magnitude decrease of the viscosity at 37° C. when compared to the 21.5° C. In other words, the increase of temperature breaks down the supramolecular structure of the gel and the molecule inside the channels becomes more exposed to the aqueous/organic environment and hence are more prompt to be released out to the ambient environment. Previous studies have shown that the drug release profiles of 5-fluorouracil-(5-FU-) loaded hydroxyapatite-gelatin (HAp-GEL) polymer composites at three different temperatures (32° C., 37° C., and 42° C.) display at each temperature, similar initial burst values, however, the release rate increases with increasing temperature. (Aydin, N. E. Effect of Temperature on Drug Release: Production of 5-FU-Encapsulated Hydroxyapatite-Gelatin Polymer Composites via Spray Drying and Analysis of In Vitro Kinetics. Int. J. Polym. Sci. 2020, 2020, 1-13.) Other researchers (Fan, J.; Zhang, H.; Yi, M.; Liu, F.; Wang, Z. Temperature induced phase transformation and in vitro release kinetic study of dihydromyricetin-encapsulated lyotropic liquid crystal. J. Mol. Liq. 2019, 274, 690-698) reported the effect of temperature (25-45° C.) on the rheological properties of several lyotropic liquid crystals encapsulating dihydromyricetin. The in vitro release results showed that the drug cumulative release percentage and release rate gradually increased as temperature rose, indicating that the release behaviors were dominated by temperature.

FIG. 11(m) depicts a graph of UV-Visible absorption spectrum of the Orange Acridine hydrochloride (OA) in H₂O in accordance with the principles of the present invention.

FIG. 11(n) depicts a graph of the diffusion kinetics of the OA through a membrane in the Franz diffusion cell for both pure OA and the OAc CDO composite in accordance with the principles of the present invention.

FIG. 11(o) depicts pictures illustrating the change of the color of the solution in the Franz diffusion cell after 4 hours for the diffusion of pure OA while the diffusion of OA from OAc-CDO is significantly slower in accordance with the principles of the present invention.

To confirm the retention of medicinal ingredients within the γ-CDO superstructure, we utilized a water soluble (solubility in water of 6 mg/ml @ 23° C.) dye such as Orange Acridine hydrochloride (OA) which is characterized (FIG. 11(m)-(o)) by its strong orange/red color and an absorption band in the visible region at 491 nm. The incorporation of OA into the γ-CDO was achieved by a slow evaporation of ethyl acetate using 6:1 mole ratio of OA:γ-CDO. In the presence of OA, the color of the γ-CDO turns uniformly from a pale yellow to dark red color on account of the intercalation of the dye inside the channels and the interstices of the superstructure of the gel. The release studies at 23° C. using the Franz diffusion cell have been achieved using 80 mg of OA⊂γ-CDO. The Franz diffusion cell is filled with 15 ml deionized H₂O and 3 ml as a carrier water phase. A fast release of the OA can be noticed for the first 5 hours which is associated to the presence of OA outside the cavity of the I-CDO and at the outer layer of the gel composite. These results are consistent to all the previous experiments. After 5 hours the OA release to the water medium is slower and reaches a plateau after 8 hours with a maximum release of 0.6%. A control experiments was achieved (FIG. 11(m)-(o)) using only the OA which revealed a fast diffusion of the dye through the membrane of the Franz diffusion cell reaching 92% diffusion within 4 hours. These results ascertain the retention of the OA inside the cavity and interstice spaces of the γ-CDO, on account of water repealing nature of the γ-CDO molecular vessels.

FIG. 11(p) depicts the temperature dependance of the OA release from the OA⊂γ-CDO composite in water in accordance with the principles of the present invention.

To test the effect of the temperature on the release of the OA, we performed (FIG. 11(p)) the release experiments at 20 and 37° C. The OA⊂γ-CDO composite (71.5 mg) was placed on the bottom of UV-visible cuvette, then filled with deionized H₂O (4 ml). The cumulative concentration of OA in the solution was monitored by UV-visible spectroscopy at a wavelength 491 nm. The OA release from OA⊂γ-CDO is faster with 0.6% release after only 2 hours and upon decreasing the temperature to 20° C. the concentration of the OA in solution remains steady indicative of the retention of the OA within the gel. The increase of temperature to 37° C. for 5 hours leads to a faster release OA reaching 1.65% then the release slows down when the temperature decreased again to 20° C. again. These results confirm the role of the rheology of the material in controlling the release of the ingredients buried within the cavities of the supramolecular channels of the γ-CDO superstructures.

### Synthesis of OSMVs

### Materials

All chemicals and reagents used in the procedures described herein were purchased from commercial suppliers (sigma Aldrich) and used without further purification. Nuclear magnetic resonance (NMR) spectra were recorded on a Bruker Avance 500 with working frequencies of 500 MHz. Chemical shifts are reported in ppm relative to the signals corresponding to the residual non-deuterated solvents (CDCl₃: d=7.26, D₂O: d=4.79). Powder X-ray diffraction (PXRD, STOE STADI-P) with Cu-Kα1 radiation (λ=1.54056 Å) was measured through transmission geometry for crystal structure analysis by scanning in the 2θ range of 10°-32° with accelerating voltage and current of 40 kV and 40 mA. Absorption of infrared radiation by the sample material is measured versus wavelength in a typical range 4000-600 cm¹ using compact Bruker FTIR spectrometer. Thermogravimetric (TGA, Netzsch) was carried out from room temperature to 400° C. at a ramping rate of 5° C. min−1 under an Ar flow. UV-Vis-NIR Spectra were recorded on a Shimadzu UV-3600 spectrophotometer. Viscosity studies have been carried at temperature range 12-60° C. using the Joan Lab viscometer (Model: JN-6502).

### Synthesis Protocols

### Preparation of the α-Cyclodextrin Oleoyl Ester (α-CDO)

FIG. 12 depicts the synthesis reaction of the oligosaccharide oleoyl esters in accordance with the principles of the present invention.

An oligosaccharide (2 g, 2.05 mmol) was dissolved in anhydrous dimethylformamide (30 mL), then pyridine (5 mL, 0.062 mol) was added. Subsequently, excess of fatty acid oil (14.84 g, 0.0495 mol) was added and the mixture was left to stir at room temperature for 24h under argon atmosphere. Excess of water was added (˜200 mL) to the DMF solution and the modified α-CD was extracted using with dichloromethane (3×40 mL). The organic layer was dried with magnesium sulfate then filtered off. The solvent was evaporated using a rotatory evaporator to offer a pale yellowish viscous oil contaminated with a white solid powder. Ethyl acetate (20 mL) was added to the crude product to solubilize the oily product while the while powder remained insoluble. After filtration and drying the solvent using rotatory evaporator then the sample was subjected to a high vacuum at 100° C. for 24 hours, a yellow pale oil was isolated. ¹H NMR spectroscopy revealed that 24 fatty acid chains have been attached to the α-CDO. While the IR spectroscopy revealed the absence of the broad peak associated to the stretching of the OH functional groups. Chemical formula: C₃₆₀H₆₃₆O₄₈, MW=5728.7 g/mol. Yield 11 g, 94% (99.99% pure). ¹H NMR: (CDCl₃, 500 MHz), SH 0.87 (54H, t); 1.28 (396H, broad); 1.65 (36H, m); 2.00 (72H, m); 2.34-2.43 (36H, t); 3.10-5.33 (broad peaks corresponding to the cyclodextrin proton resonance); 5.33 (36H, s). IR (νₘₐₓ/cm⁻¹): 3009 s, 2918 m, 2851 m, 1711 s, 1744 m (C═O ester).

FIG. 13 depicts the ¹H NMR spectrum of the α-CDO in CDCl₃ at 298K in accordance with the principles of the present invention.

### Preparation of the β-Cyclodextrin Oleoyl Ester (β-CDO-1)

β-Cyclodextrin (2 g, 1.76 mmol) was dissolved in anhydrous dimethylformamide (30 mL), then pyridine (5 mL, 0.062 mol) was added. Subsequently, excess of fatty acid oil (12.72 g, 0.0425 mol) was added and the mixture was left to stir at room temperature for 24 h under argon atmosphere. Excess of water was added (˜200 mL) to the DMF solution and the modified β-CD was extracted using with dichloromethane (3×40 mL). The organic layer was dried with magnesium sulfate then filtered off. The solvent was evaporated using a rotatory evaporator to offer a pale yellow/orange viscous oil contaminated with a white solid powder. Ethyl acetate (20 mL) was added to the crude product to solubilize the oily product while the while powder remained insoluble. After filtration and drying the solvent using rotatory evaporator then the sample was subjected to a high vacuum at 100° C. for 24 hours, a yellow pale oil was isolated. ¹H NMR spectroscopy revealed that 24 fatty acid chains have been attached to the β-CDO. While the JR spectroscopy revealed the absence of the broad peak associated to the stretching of the OH functional groups. Chemical formula: C₄₂₀H₇₄₂O₅₆, MW=6683.5 g/mol, Yield 11.5 g, 98% (98% pure). ¹H NMR: (CDCl₃, 500 MHz), δH 0.88 (63H, t); 1.28 (462H, broad); 1.63 (42H, m); 2.00 (84H, t); 2.25-2.50 (42H, m); 3.11-5.23 (broad peaks corresponding to the cyclodextrin proton resonance); 5.33 (42H, s). IR (νₘₐₓ/cm⁻¹): 3009 s, 2918 m, 2851 m, 1711 s, 1744 m (C═O ester).

FIG. 14 depicts the ¹H NMR spectrum of the β-CDO in CDCl₃ at 298K in accordance with the principles of the present invention.

### Preparation of the β-Cyclodextrin Oleoyl Ester (β-CDO-2)

β-Cyclodextrin (2 g, 1.76 mmol) was dissolved in anhydrous dimethylformamide (30 mL), then pyridine (5 mL, 0.062 mol) was added. Subsequently, excess of fatty acid oil (8 g, 0.0266 mol) was added and the mixture was left to stir at room temperature for 24 h under argon atmosphere. Excess of water was added (˜200 mL) to the DMF solution and the modified β-CD was extracted using with dichloromethane (3×40 mL). The organic layer was dried with magnesium sulfate then filtered off. The solvent was evaporated using a rotatory evaporator to offer a pale yellow/orange viscous oil contaminated with a white solid powder. Ethyl acetate (20 mL) was added to the crude product to solubilize the oily product while the while powder remained insoluble. After filtration and drying the solvent using rotatory evaporator then the sample was subjected to a high vacuum at 100° C. for 24 hours, a yellow pale oil was isolated. ¹H NMR spectroscopy revealed that 14 fatty acid chains have been attached to the β-CDO-2. Chemical formula: C₂₉₄H₅₀₄O₄₉, MW=4823 g/mol, Yield 11.5 g, 98% (98% pure). ¹H NMR: (CDCl₃, 500 MHz), δH 0.88 (42H, t); 1.28 (364H, broad); 1.63 (28H, m); 2.00 (56H, t); 2.25-2.50 (35H, m); 3.11-5.23 (broad peaks corresponding to the cyclodextrin proton resonance); 5.33 (28H, s).

### Preparation of the γ-Cyclodextrin Oleoyl Ester (γ-CDO)

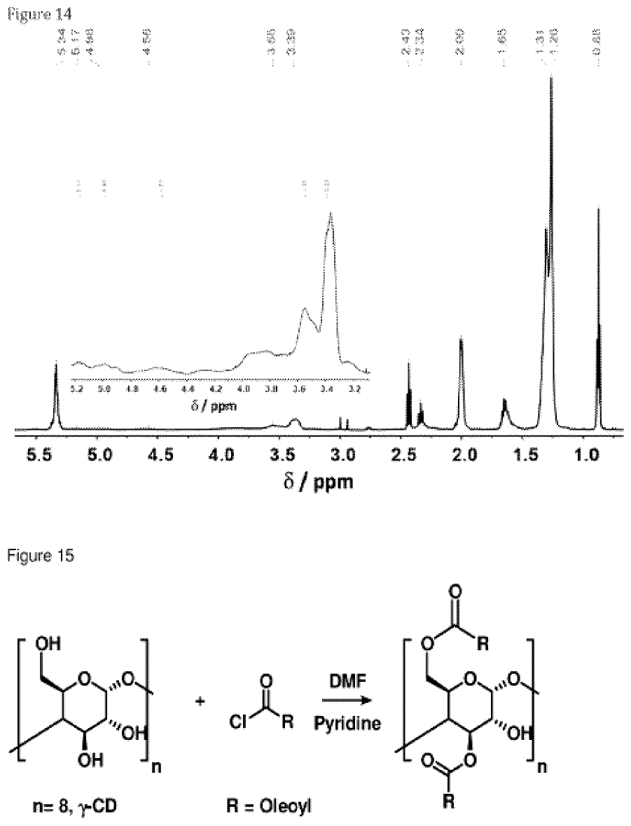

FIG. 15 depicts the synthesis of the γ-CD oleoyl esters in accordance with the principles of the present invention.

γ-Cyclodextrin (2 g, 1.54 mmol) was dissolved in anhydrous dimethylformamide (30 mL), then pyridine (5 mL, 0.062 mol) was added. Subsequently, excess of oleoyl chloride (11.2 g, 0.0375 mol) was added and the mixture was left to stir at room temperature for 24 h under argon atmosphere. Excess of water was added (˜200 mL) to the DMF solution and the modified γ-CD was extracted using with dichloromethane (3×40 mL). The organic layer was dried with magnesium sulfate then filtered off. The solvent was evaporated using a rotatory evaporator to offer a pale yellowish viscous oil contaminated with a white solid powder. Ethyl acetate (20 mL) was added to the crude product to solubilize the oily product while the while powder remained insoluble. After filtration and drying the solvent using rotatory evaporator then the sample was subjected to a high vacuum at 100° C. for 24 hours, a yellow pale viscous liquid was isolated. ¹H NMR spectroscopy revealed that 16 fatty acid chains have been attached to the γ-CDO. This is confirmed by the JR spectroscopy which reveals the persistence of the broad peak associated to the stretching of the OH functional groups. Chemical formula: C₃₃₆H₅₉₂O₅₆, MW=5524.35 g/mol. Yield 8.2 g, 96% (99.99% pure). ¹H NMR: (CDCl₃, 500 MHz), δ_{H} 0.90 (48H, t); 1.29 (352H, broad); 1.65 (32H, m); 2.03 (64H, t); 2.32 (32H, m); 3.16-5.19 (broad peaks corresponding to the cyclodextrin proton resonance); 5.36 (32H, s). IR (νₘₐₓ/cm⁻¹): 3330 w, 3010 s, 2925 m, 2851 m, 1710 s, 1740 s (C═O ester). Thermogravimetric analysis (TGA): 30-260° C. (onset T=192.3° C., weight loss=25%), 260-400° C. (onset T=279.1° C., weight loss=45%).

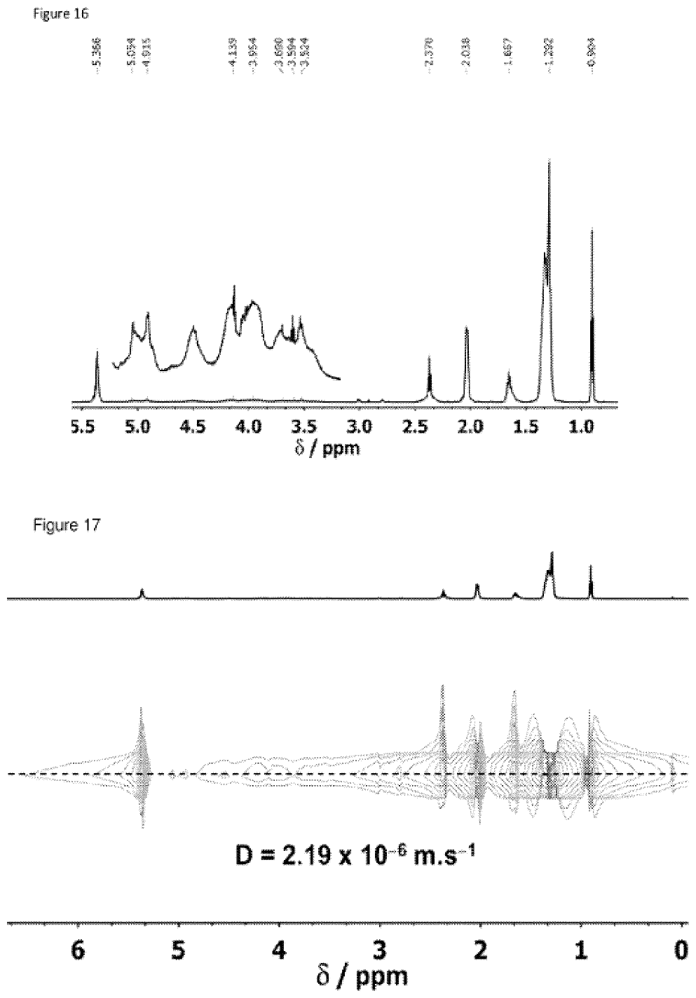

FIG. 16 depicts the ¹H NMR spectrum of the γ-CDO in CDCl₃ at 298K in accordance with the principles of the present invention.

FIG. 17 depicts 2D NMR DOSY spectrum of the γ-CDO in CDCl₃ at 298K in accordance with the principles of the present invention.

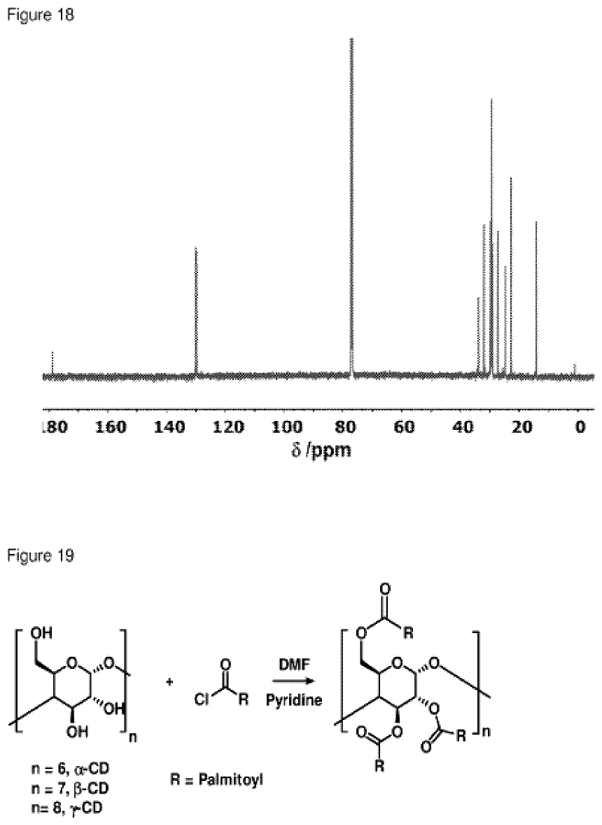

FIG. 18 depicts the ¹³C NMR spectrum of the γ-CDO in CDCl₃ at 298K in accordance with the principles of the present invention.

### Preparation of the Oligosaccharide Palmitoyl Esters

FIG. 19 depicts the Synthesis of the cyclodextrin palmitoyl esters in accordance with the principles of the present invention.

### Preparation of the α-Cyclodextrin Palmitoyl Ester (α-CDP)

An oligosaccharide (2 g, 2.05 mmol) was dissolved in anhydrous dimethylformamide (30 mL), then pyridine (5 mL, 0.062 mol) was added. Subsequently, excess of fatty acid oil (14.84 g, 0.0495 mol) was added and the mixture was left to stir at room temperature for 24 h under argon atmosphere. Excess of water was added (˜200 mL) to the DMF solution and the modified α-CD was extracted using with dichloromethane (3×40 mL). The organic layer was dried with magnesium sulfate then filtered off. The solvent was evaporated using a rotatory evaporator to offer a pale yellowish viscous oil contaminated with a white solid powder. Ethyl acetate (20 mL) was added to the crude product to solubilize the oily product while the while powder remained insoluble. After filtration and drying the solvent using rotatory evaporator then the sample was subjected to a high vacuum at 100° C. for 24 hours, a yellow pale oil was isolated. ¹H NMR spectroscopy revealed that 24 fatty acid chains have been attached to the α-CDP. While the JR spectroscopy revealed the absence of the broad peak associated to the stretching of the OH functional groups. Chemical formula: C₃₂₄H₆₀₀O₄₈, MW=5260.45 g/mol. Yield 10.5 g, 97% (96% pure). ¹H NMR: (CDCl₃, 500 MHz), δ_{H} 0.86 (54H, t); 1.23 (468H, broad); 1.63 (36H, m); 2.32-2.42 (36H, t); 3.20-5.68 (broad peaks corresponding to the cyclodextrin proton resonance). IR (νₘₐₓ/cm⁻¹): 2919 m, 2847 m, 1743 s (C═O ester), 1711 m.

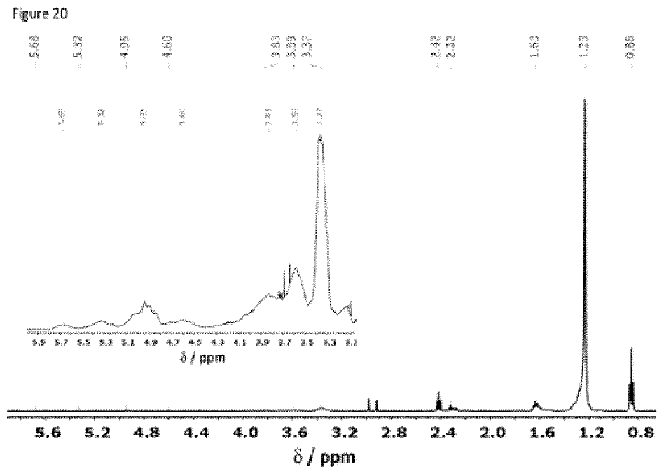

FIG. 20 depicts the ¹H NMR spectrum of the α-CDP in CDCl₃ at 298K in accordance with the principles of the present invention.

### Preparation of the β-Cyclodextrin Palmitoyl Ester (β-CDP)

An oligosaccharide (2 g, 1.76 mmol) was dissolved in anhydrous dimethylformamide (30 mL), then pyridine (5 mL, 0.062 mol) was added. Subsequently, excess of fatty acid oil (12.72 g, 0.0425 mol) was added and the mixture was left to stir at room temperature for 24 h under argon atmosphere. Excess of water was added (˜200 mL) to the DMF solution and the modified β-CD was extracted using with dichloromethane (3×40 mL). The organic layer was dried with magnesium sulfate then filtered off. The solvent was evaporated using a rotatory evaporator to offer a pale-yellow solid. Ethyl acetate (20 mL) was added to the crude product to and after filtration and drying the solvent using rotatory evaporator then the sample was subjected to a high vacuum at 100° C. for 24 hours, a yellow pale solid was isolated. ¹H NMR spectroscopy revealed that 16 fatty acid chains have been attached to the β-CDP. While the IR spectroscopy revealed the absence of the broad peak associated to the stretching of the OH functional groups. Chemical formula: C₃₇₀H₇₀₀O₅₆, MW=6141.7 g/mol. Yield 10.4 g, 96% (97% pure). ¹H NMR: (CDCl₃, 500 MHz), δ_{H} 0.88 (63H, t); 1.25 (546H, broad); 1.67 (42H, m); 2.32-2.44 (42H, t); 3.23-5.51 (broad peaks corresponding to the cyclodextrin proton resonance). IR (νₘₐₓ/cm⁻¹): 2919 m, 2847 m, 1743 s (C═O ester), 1711 m. Thermogravimetric analysis (TGA): 30-390° C. (onset T=192.3° C., weight loss=90%).

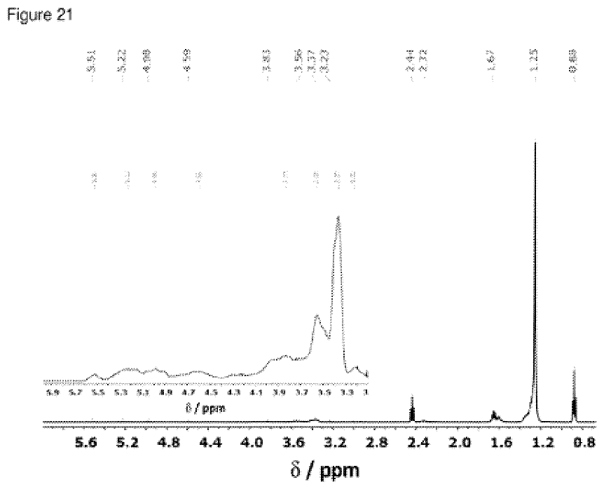

FIG. 21 depicts the ¹H NMR spectrum of the Oligosaccharide fatty acid ester in CDCl₃ at 298K in accordance with the principles of the present invention.

### Preparation of the γ-Cyclodextrin Palmitoyl Ester (γ-CDP)

γ-Cyclodextrin (2 g, 1.54 mmol) was dissolved in anhydrous dimethylformamide (30 mL), then pyridine (5 mL, 0.062 mol) was added. Subsequently, excess of palmitoyl chloride (10.3 g, 0.0375 mol) was added and the mixture was left to stir at room temperature for 24 h under argon atmosphere. Excess of water was added (˜200 mL) to the DMF solution and the modified γ-CD was extracted using with dichloromethane (3×40 mL). The organic layer was dried with magnesium sulfate then filtered off. The solvent was evaporated using a rotatory evaporator to offer a pale yellowish solid. Ethyl acetate (20 mL) was added to the crude product and after filtration and drying the solvent using rotatory evaporator then the sample was subjected to a high vacuum at 100° C. for 24 hours, a yellow pale solid was isolated. ¹H NMR spectroscopy revealed that 16 fatty acid chains have been attached to the γ-CDP. Chemical formula: C₃₀₄H₅₆₀O₅₆, MW=5108.10 g/mol. Yield 7.6 g, 96% (99.99% pure). ¹H NMR: (CDCl₃, 500 MHz), δ_{H} 0.88 (48H, t); 1.28 (416H, broad); 1.65 (32H, t); 2.36 (32H, m); 3.52-5.05 (broad peaks corresponding to the cyclodextrin proton resonance). IR (νₘₐₓ/cm⁻¹): 2919 m, 2847 m, 1743 s (C═O ester), 1711 m. Thermogravimetric analysis (TGA): 50-260° C. (onset T=192.5° C., weight loss=20%), 260-380° C. (onset T=340° C., weight loss=45%).

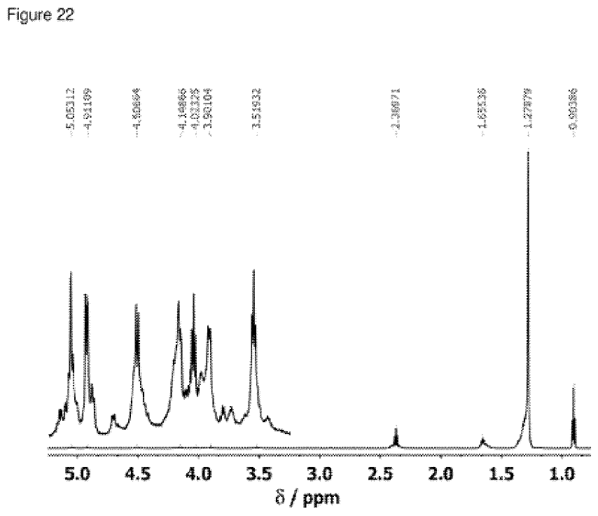

FIG. 22 depicts the ¹H NMR spectrum of the γ-CDP ester in CDCl₃ at 298K. (PCD) in accordance with the principles of the present invention.

FIG. 23 depicts the 2D NMR DOSY spectrum of the γ-CDP in CDCl₃ at 298K PCD in accordance with the principles of the present invention.

### Preparation of Host-Guest Inclusion Complexes

Loading medicinal active ingredients into OSMV (600 g) may be achieved through a slow evaporation of organic solvents. The OSMVs have been solubilized in ethyl acetate and add in small portions 80 g of Niacinamide. The mixture may be sonicated at 50° C. for 1h until majority of niacinamide is solubilized. The little precipitate of niacinamide was filtered off then solubilized in water (30 mL) and added to the ethyl acetate solution. After a slow evaporation of solvents, a host-guest complexes of OSMV and active ingredients has been formed. Similar procedure was used to incorporate allantoin, ferulic acid, resveratrol, and vitamin C inside the OSMV. Different classes of ingredients may be sorted into 3 groups: pharmaceutical, cosmetic skin care, and cosmetic fragrance. Within each, subclasses may be present. For example, the limitations for selecting the active ingredient may be the size, and secondarily, the ability to dissolve the active ingredient in non-aqueous solvents will improve the loading efficacy.

FIG. 24 depicts the thermogravimetric analysis of the α-CDP in accordance with the principles of the present invention.

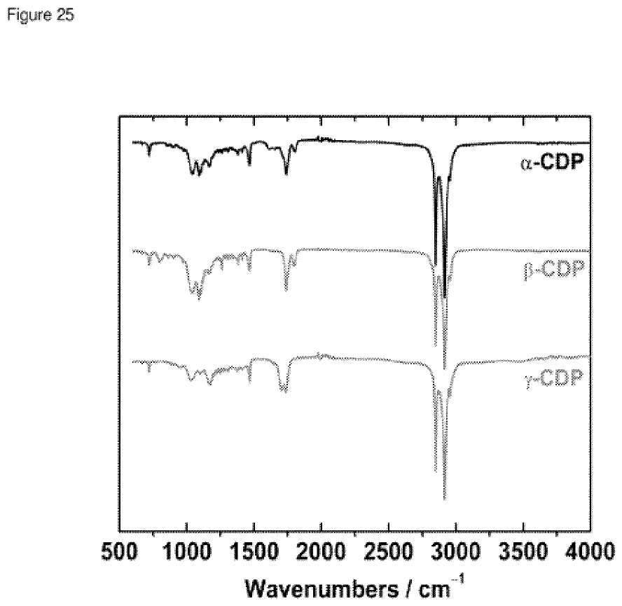

FIG. 25 depicts the FT-IR spectra of α-CDP, β-CDP and γ-CDP in accordance with the principles of the present invention.

FIG. 26 depicts the FT-IR spectra of α-CDO. β-CDO and γ-CDO in accordance with the principles of the present invention.

### Topical Application Through OSMVs

The concept of oral delivery may be similar in all respects to topical delivery, except instead of skin enzymes, stomach enzymes may break down the OSMV. Furthermore, internal body temperature may be higher and OSMV structure adjusted accordingly. Water dissolution in the stomach may tend to speed up the release since the water may penetrate inside the OSMV and may facilitate faster out diffusion of the trapped ingredients. In terms of release profile and concentrations, the design may be similar to topical delivery and may depends on the specific active ingredient used.

Embodiments of the present invention include topical or transdermal delivery of cosmetic active ingredients through OSMVs. OSMV slow, controlled release delivery may be appropriate when you need to deliver an active ingredient for as long as possible (for continuous treatment) and at controlled rate (to prevent overexposure, irritation/sensitization). A cosmetic active ingredient may have a therapeutic function to improve, heal, or cure cosmetic or dermatological conditions. Topical or transdermal delivery of cosmetic active ingredients may be an important component in the overall efficacy of a cosmetic production formulation. The ingredient needs to be delivered to the skin surface at the right concentration for a sufficient period of time using Organic Super Molecular Vessels (OSMVs). Such concentration may depend on the actual ingredient used. For example, for retinol, a moderate strength may be 0.04% to 0.1%. Higher concentration may be used for stronger treatment but may cause dermatitis for some users. OSMV may pack 0.5% and release over 5× longer time and be effective a 0.1% dosage but for 5× longer treatment duration. In some embodiments, active ingredient delivery may be useful to continue until the next time the user washes the area. Thus, embodiments of the present invention include use of OSMVs to deliver an active ingredient at a predetermined concentration. Furthermore, the OSMVs may be used to deliver the active ingredient over a predetermined sustained period of time. The concentration may comprise the recommended clinical concentration. The time period may not necessarily be controllable for creams currently on the market. Manufacturers put the concentration of the active ingredient in the cream, and when the active ingredients penetrate the skin and are depleted, then that's the end of the treatment. The only way to extend the treatment of presently marketed creams is to re-apply. OSMVs of the present invention may give the capability to store a surplus amount of the active ingredient like a reservoir to slowly release the active ingredient for a sustained period of time.

OSMVs are stable and water insoluble macromolecules which are assembled according to the procedure set forth above to form porous supramolecular tubular structures known as OSMVs that can encapsulate active ingredients' molecules to enhance delivery thereof to the skin surface at optimal concentrations for prolonged periods of time. Embodiments of the present invention tune the OSMV chemical composition by type and ratio of cyclodextrins and fatty acid esters to control the cavity size formed, as well as loading and delivery efficacy for different corresponding active ingredients. OSMV may comprise a composite material of oligosaccharide (e.g., CDs) and fatty acid chains interconnected together to form a “web.” The CDs can be viewed as the “nodes” and the fatty acid chains as the “links.” Modifying the number of nodes and links may affect the density of the network while will affect the cavity geometry. For bigger molecules, bigger cavity space may be desirable. Therefore, a lower density of fatty acids may be used with higher density of nodes. In addition, while saturated aliphatic chains may form crystalline solids, unsaturated aliphatic chains may form semi-crystalline organogels of high viscosity at room temperature. The semi-crystalline organogel viscosity may decrease dramatically at the skin temperature, such as 37 degrees Celsius (98.6 degrees Fahrenheit), which may trigger the drug release upon usage of the products.

One or more active ingredients' molecules can be loaded into the OSMV in non-aqueous solvent alone or in a mixture of non-aqueous solvent and water at different ratios depending on the solubility of the active ingredient in the aqueous and non-aqueous solvents. Ratios here may be referred to as the water:non-aqueous solvent ratios. If the active ingredient is only water-soluble, then a 10% or 20% or water may be required to dissolve it enough in the mixed solvent to diffuse/load into the OSMV. If the active ingredient is highly soluble in an organic solvent like ethanol, then in this case, 100% ethanol solvent may be used. The active ingredient may be dissolved in 100% ethanol and the unloaded/empty OSMV may be submerged in the solution to let the dissolved ingredient diffuse and enter/load into the OSMV. The solvent may then evaporated or the loaded OSMV may be filtered to get rid of the solvent. Loading by diffusion may typically take the OSMV remaining in the saturated solution for 24 hours.

Once the active ingredients diffuse into the OSMV cavities, the excess solvent can be filtered or evaporated. After removal of the solvent, the overall assembly of the OSMV will not change significantly, but the cavity of the OSMV will be loaded with active ingredients. The active ingredients are hosted in (i) The cavity of the OSMV and (ii) the interstice space between the fatty acid ester chains comprising the OSMV super structure. The activated OSMV can then be dispersed in either an aqueous or non-aqueous medium or combination thereof to form various emulsion or non-emulsion type formulations, including but not limited to serum, lotion, gel, cream, suspension, liquid, or powder. Active ingredients' molecules stored outside the OSMV, readily dispersed throughout the carrier medium, will be the first to reach the skin for an immediate efficacy. “Free” active ingredients' molecules are defined as the active ingredients not encapsulated within OSMV and are freely solubilized/dispersed through the carrier medium of the formulation. The carrier medium can be water, oil, and/or combination of either one with or without one or more other carrier solvents or fillers. In contrast, encapsulated active ingredients' molecules stored inside the OSMV have to first diffuse out of the OSMV cavities before they are “free” to reach the skin barrier. These active ingredients' molecules are initially trapped inside the OSMV pores and cavities.

Once the activated OSMV particles are applied onto the skin, the higher biological temperature may lead to the decrease of the viscosity of the OSMV. In turn, the OSMVs may gradually unwind as the hydrophobic components of the OSMV adsorb onto the skin. This unwinding may widen the internal pores and cavities and create continuous channels that may facilitate the encapsulated active ingredients to diffuse and escape the OSMV structure and subsequently reach the skin barrier. In addition, the OSMV may be digested by the skin enzymes to liberate the cyclodextrin macrocycle and the fatty acid components. For example, skin enzymes may include esterase enzymes located in keratinocytes in the epidermis layer, the outermost layer of the skin. The resulting reactions products may have great benefits for the skincare. Enzymes may hydrolyze the OSMV and release the fatty acid components attached to the OSMV. The fatty acids may be broken off from the OSMV. OSMV may be comprised of oligosaccharides (CDs in this case) and fatty acids. The fatty acid component is broken off and liberated and can have benefits for the skin.

For a given active ingredient and carrier medium, by optimizing the ratio and concentration of active ingredient inside and outside the OSMV as well as the concentration and size of OSMV, a desired release profile may be created. The release profile may be defined as the amount of the active ingredient that reaches the skin membrane barrier vs time. The release profile may depend on the active ingredient used. For embodiments in which retinol is the active ingredient, moderate concentration at 0.1% may be desirable but for 5× longer treatment duration. Thus, 0.5% retinol may be loaded but perhaps at a retinol: OSMV loading ratio of 10% (instead of 40%) so the retinol may be sparser within the OSMV. This may ensure slowest release of the retinol. The actual release time may be relative. For example, if it takes 2 hours for pure 0.1% retinol in a viscous cream to fully absorb into the skin; it may take 10 hours for all the 0.5% retinol trapped within OSMV to fully absorb into the same viscous base cream. This may translate to an effective concentration of 0.1% for an effective duration of 10 hours compared to an effective concentration of 0.1% for only 2 hours.

Since OSMVs do not degrade in a mild aqueous or non-aqueous media, not only can it be dispersed in aqueous, non-aqueous, or emulsions, OSMVs can also be loaded with both hydrophobic and hydrophilic ingredients. In addition, OSMV multi-level cavities and size tunability enables OSMVs to house a wide variety of active ingredients, ranging in size from small molecules to polymers. In 100% water, OSMV will tend to agglomerate and can increase in particle size from 100 nm to 500 um. Due to the ability to control the cavity and particle size of OSMV, active ingredients with an average particle size up to about 10 million Daltons can be encapsulated. While several small molecules such as niacinamide and vitamin C can be encapsulated within the cavity, linear polymers such hyaluronic acid can form polyrotaxane supramolecular structures.

Typical loading ratios range from about 0.1 mg to about 0.6 mg of the active ingredient per 1 mg of OSMV. These ratios of active ingredient to OSMV does not include the amount of active ingredient purposely kept outside the OSMV in the formulation. Within the loading ratios stated above, the formulations may significantly leverage the benefit of OSMV. For example, at higher loading ratios, there may be incremental benefit of having any more active ingredient stored inside of OSMV rather than outside because the excess active ingredient trapped within the interstice of the fatty acid chains comprising the outer layer of the OSMV particle will not be sufficiently trapped. This excess active ingredient near the outer layer of the OSMV particle may only release from the OSMV at a slightly slower rate compared to the “free” active ingredients.

OSMV can encapsulate active ingredients that can be either water insoluble with water solubility less than 1 g/L at 25° C. or water soluble with water solubility at least 1 g/L at 25 C. Noteworthy, the solubility of the OSMV in non-aqueous solvents allows the encapsulation of the water-insoluble active ingredients using 100% non-aqueous solvents such as glycerin, oils, and organic solvents. For the active ingredient which are not soluble in non-aqueous solvents, a mixture of non-aqueous water-miscible solvent/water at ratios ranging from 90/10 to 10/90 can be utilized to achieve an efficient encapsulation of the active ingredients. Although possible, a 100% water solvent is not necessarily recommended because the OSMV will tend to agglomerate and decrease the loading efficiency. The water-soluble active ingredients may become soluble in non-aqueous solvents after encapsulation within the OSMV, allowing therefore, not only a facile incorporation of the composites within the cosmetic formulations but also offer a possibility to investigate the physical properties of the water-soluble active ingredients in non-aqueous medias. OSMV can be stably dispersed in both O/W (oil in water) and W/O (water in oil) emulsions at various water:oil ratios even up to 100% water or 100% oil (completely anhydrous).

For an emulsion formulation, OSMVs can be solubilized in the oil phase prior to the emulsification with the water phase. Water-soluble and/or oil-soluble active ingredients can be securely housed inside the OSMV within the oil phase. By further confining hydrophilic ingredients within OSMV surrounded in an oil phase, the release of the hydrophilic ingredient can be further slowed down compared to housing the hydrophilic ingredient within OSMV surround in the water phase.

### Examples

The ability of OSMVs to host and the slow release the active ingredients of two common active ingredients, resveratrol and niacinamide, were investigated. The experimental data demonstrate the breadth of OSMV capability to carry both oil-soluble (resveratrol) and water-soluble (niacinamide) ingredients in an aqueous (water) media. OSMV can be incorporated in a wide variety of formulations due to its ability to incorporate various types of hydrophobic or hydrophilic active ingredient in various type of carrier media composed of aqueous or non-aqueous or mixtures thereof.

Initially, the retention of the active ingredients within the OSMV in water were studied. The drug release studies were conducted on the Resveratrol⊂OSMV (5 mg) host-guest complex in deionized H₂O (3 ml) using UV-visible spectroscopy. The resveratrol has a strong absorption band are 315 nm (FIG. 9a) facilitating monitoring the concentration of the resevertarol released to the solution. Upon addition of water to the Resveratrol⊂OSMV complex, the intensity of the absorbance is very small (0.46) and the intensity increases slightly after 100 min (FIG. 9b) indicating the ability of the OSMV to enhance the retention of the medicinal active ingredients in aqueous media. After 100 min, the resveratrol release is accelerated and the maximum absorbance reaches 4.8 at 700 min. The one fold increase of the reseveratrol concentration in water after 12 hours, confirm the ability of the OSMVs superstruture to host and release medicinal active ingredients.

The improvement of transdermal delivery using OSMV is demonstrated using a transdermal diffusion instrument. The instrument can measure the speed and degree of penetration of active ingredients from a topical formulation through a synthetic membrane simulating the skin. The vertical diffusion cells have 15 mL volume bottom receptor chamber with a magnetic stirrer and top a donor chamber with a synthetic membrane separating the bottom and top chambers. The test formulation is placed in the top chamber directly on top of the membrane. The amount of active ingredient that traverses the synthetic membrane and into the receptor chamber is quantified by sampling the receptor solvent at specific times while also ensuring new solvent is added to maintain a fixed 15 mL receptor volume. The concentration of active ingredient analyte in the receptor solvent sample is quantified using a UV-Vis spectrometer. The diffusion cells are placed in a water bath maintained at a constant 32° C. temperature. Samples were taken every 10 min to quantify how much active ingredient penetrated the membrane over time. Strat-M membranes were used in these experiments. Strat-M membrane is a synthetic, non-animal-based membrane model for transdermal diffusion testing that is predictive of diffusion in human skin. It is designed for screening of active pharmaceutical ingredients (API) and cosmetic actives.

In the first set of experiments, 1 mg of resveratrol is incorporated into 4 mg OSMV and pure water was added. The amount of resveratrol that is released by the OSMV and crosses the artificial membrane was monitored over time using UV-visible spectroscopy. The results are consistent with the previous UV-vis experiments showing a slow release of the resveratrol for a period of at least 12 hours. In the absence of the OSMV the resveratrol (1 mg) is soluble in water and traverse the membrane in 1 hour. Similar results have been obtained with niacinamide incorporated within the OSMV. The high solubility of niacinamide in water leads a fast transdermal absorption while in the presence of the OSMV, the Niacinamide is released gradually to the aqueous media allowing a longer time efficacy on the skin. Niacinamide may be similar to the release profile illustrated in FIG. 11b, wherein 700 minutes may start flattening and may reach almost full cumulative release.

In conclusion, encapsulation of active ingredient into supramolecular vessels has tremendous benefits to enhance the chemical stability of the active ingredients, avoid harmful high concentrations as a result of the slow release of the active ingredients and make the chemicals available for a prolong period of time.

Embodiments of the present invention may include an organic super molecular vessel (OSMV), including a cyclodextrin host comprising a polymer chain of structural units shaped as a frustoconical annulus formed around a cavity defined from a first end to a second end; and a fatty acid ester bound to at least one of the structural units. The cyclodextrin may be an α-cyclodextrin, a ß-cyclodextrin, a γ-cyclodextrin. The fatty acid may be at least one of the following: palmitoyl, oleoyl, oleic acid, linoleic acid, linolenic acid, stearic acid, a congener thereof. In some embodiments, the fatty acid may consist of palmitoyl, oleoyl, oleic acid, linoleic acid, linolenic acid, stearic acid, a congener thereof. A guest molecule may be positioned at least partially within the cavity of the OSMV. The guest molecule may further comprise a pain reliever. The pain reliever comprises at least one of: lidocaine, naproxen, lansoprazole, ibuprofen, acetaminophen, diclofenac, oxycodone, fentanyl, hydrocodone. The guest molecule may comprise a chemotherapeutic drug. The chemotherapeutic drug may comprise at least one of: chemotherapeutic drugs letrozole, sonidegib, ruxolitinib, abiraterone, altretamine, Palbociclib, procarbazine, and sunitinib. An opening at the first end may be the same size as the opening at the second end.

A pore size of a spherical cavity formed of six OSMVs may be 1.7 nm. A pore size of a cavity formed between two α-CD may be about 0.57 nm, β-CD may be about 0.78 nm, and γ-CD may be about 0.95 nm. Embodiments of the present invention may include a structure formed of at least two of the OSMVs, wherein the OSMVs stack by association of the one or more corresponding ester-bound fatty acid functional groups. An interlamellar distance between the at least two OSMVs may be about 3.7-4.0 nm.

Further embodiments may include a method of forming an OSMV, including dissolving cyclodextrin in dimethylformamide under inert conditions to form a solution, adding pyridine to the solution, adding excess of a fatty acid oil to form a mixture, adding excess water, and extracting with dichloromethane. The cyclodextrin may be one of the following: an α-cyclodextrin, a ß-cyclodextrin, a γ-cyclodextrin. The fatty acid oil may be one of the following: palmitoyl, oleoyl, oleic acid, linoleic acid, linolenic acid, stearic acid, a congener thereof. The mixture may be stirred at room temperature for 24 hours under inert atmosphere.

Further embodiments of the present invention include a method of administering an active ingredient/drug, including preparing an OSMV, loading the OSMV with the active ingredient, applying the loaded OSMV to a patient, and raising the application to biological temperature.

The loaded OSMV may be raised to biological temperature by applying the loaded OSMV to a patient. The application may be applied to the skin of a patient topically, and wherein the active ingredient may be an active cosmetic ingredient. The cosmetic active ingredient may be one of the following: water-soluble vitamin B, niacinamide, ginseng extract, gotu kola extract, green tea extract, oil-soluble resveratrol. Raising the temperature may include dosing the application orally, and wherein the active ingredient is a pharmaceutical. The pharmaceutical may include one of the following: oil-soluble resveratrol, vitamin E, tocopherol, and water-soluble vitamin C, magnesium ascorbyl phosphate.

Although the invention has been discussed with reference to specific embodiments, it is apparent and should be understood that the concept can be otherwise embodied to achieve the advantages discussed. In this regard, the foregoing description of the systems and methods is presented for purposes of illustration and description.

Furthermore, the description is not intended to limit the invention to the form disclosed herein. Accordingly, variants and modifications consistent with the following teachings, skill, and knowledge of the relevant art, are within the scope of the present invention. The embodiments described herein are further intended to explain modes known for practicing the invention disclosed herewith and to enable others skilled in the art to utilize the invention in equivalent, or alternative embodiments and with various modifications considered necessary by the particular application(s) or use(s) of the present invention.

Although the invention has been discussed with reference to specific embodiments, it is apparent and should be understood that the concept can be otherwise embodied to achieve the advantages discussed. The preferred embodiments above have been described primarily as structures and methods for ordered supramolecular structures for extended release of active ingredient. In this regard, the foregoing description of the structures and methods is presented for purposes of illustration and description.

Furthermore, the description is not intended to limit the invention to the form disclosed herein. Accordingly, variants and modifications consistent with the following teachings, skill, and knowledge of the relevant art, are within the scope of the present invention. The embodiments described herein are further intended to explain modes known for practicing the invention disclosed herewith and to enable others skilled in the art to utilize the invention in equivalent, or alternative embodiments and with various modifications considered necessary by the particular application(s) or use(s) of the present invention.

What is claimed is:

1. An organic supramolecular structure called organic super molecular vessel (OSMV), comprising:
a cyclodextrin host comprising a polymer chain of structural units shaped as a frustoconical annulus formed around a cavity defined from a first end to a second end; and
a fatty acid ester bound to at least one of the structural units.

2. The organic super molecular vessel according to claim 1, wherein the cyclodextrin is one of the following: an α-cyclodextrin, a ß-cyclodextrin, a γ-cyclodextrin.

3. The organic super molecular vessel according to claim 1, wherein the fatty acid comprises at least one of the following: palmitoyl, oleoyl, oleic acid, linoleic acid, linolenic acid, stearic acid, or a congener thereof.

4. The organic super molecular vessel according to claim 1, further comprising:
an active ingredient guest molecule positioned at least partially within the cavity of the organic super molecular vessel.

5. The organic super molecular vessel of claim 4, wherein the guest molecule comprises an active pharmaceutical ingredient.

6. The organic super molecular vessel of claim 4, wherein the guest molecule comprises an at least one of the following: oil-soluble resveratrol, vitamin E, tocopherol, water-soluble vitamin C, or a congener thereof.

7. The organic super molecular vessel of claim 4, wherein the guest molecule comprises an active cosmetic ingredient.

8. The organic super molecular vessel of claim 7, wherein the cosmetic active ingredient comprises one of the following: water-soluble vitamin B, niacinamide, ginseng extract, gotu kola extract, and green tea extract, oil-soluble resveratrol, or a congener thereof.

9. The organic super molecular vessel of claim 5, wherein the guest molecule comprises a chemotherapeutic drug.

10. The organic super molecular vessel of claim 4, wherein the guest molecule comprises an active fragrance ingredient.

11. The organic super molecular vessel according to claim 1, wherein an opening at the first end is the same size as the opening at the second end.

12. The organic super molecular vessel according to claim 1, wherein a pore size of a spherical cavity formed of six organic super molecular vessels is 1.7 nm.

13. The organic super molecular vessel according to claim 1, wherein a pore size of a cavity formed between two α-CD is about 0.57 nm, β-CD is about 0.78 nm, and γ-CD is about 0.95 nm.

14. A structure formed of at least two of the organic super molecular vessels of claim 1, wherein the organic super molecular vessels stack by association of the one or more corresponding ester-bound fatty acid functional groups.

15. The structure of claim 14, wherein an interlamellar distance between the at least two organic super molecular vessels is about 3.7-4.0 nm.

16. A method of forming an organic super molecular vessel, comprising:
dissolving cyclodextrin in dimethylformamide under inert conditions to form a solution;
adding pyridine to the solution;
adding excess of a fatty acid oil to form a mixture;
adding excess water; and
extracting with dichloromethane.

17. The method of claim 16, wherein the cyclodextrin is one of the following: an α-cyclodextrin, a ß-cyclodextrin, a γ-cyclodextrin.

18. The method of claim 16, wherein the fatty acid oil is one of the following: palmitoyl, oleoyl, oleic acid, linoleic acid, linolenic acid, stearic acid, or a congener thereof.

19. The method of claim 16, further comprising;
stirring the mixture at room temperature for 24 hours under inert atmosphere.

20. A method of administering an active ingredient/drug, comprising:
preparing an organic super molecular vessel;
loading the organic super molecular vessel with the active ingredient;
applying the loaded organic super molecular vessel to a patient;
raising the application to biological temperature.

21. The method of claim 20, wherein preparing the organic super molecular vessel further comprises:
raising the loaded organic super molecular vessel to biological temperature by applying the loaded organic super molecular vessel to a patient.

22. The method of claim 20, wherein raising the temperature comprises applying the application to the skin of a patient topically, and
wherein the active ingredient is an active cosmetic ingredient.

23. The method of claim 20, wherein raising the temperature comprises applying the application to the skin of a patient topically, and
wherein the active ingredient is an active fragrance ingredient.

24. The method of claim 20, wherein raising the temperature comprises dosing the application orally, and wherein the active ingredient is an active pharmaceutical ingredient.
